SEQUENCE LISTING (6 sequences)

SEQ ID NO: 1 - DNA, 20 bp, Artificial Sequence
  SYNTHETIC PRIMER
ctcgtaatatcggtatcatg

SEQ ID NO: 2 - DNA, 20 bp, Artificial Sequence
  SYNTHETIC PRIMER
taagggtcagtcataacttt

SEQ ID NO: 3 - DNA, 21 bp, Artificial Sequence
  SYNTHETIC PRIMER
tgatcgtttacaagctaacgc

SEQ ID NO: 4 - DNA, 21 bp, Artificial Sequence
  SYNTHETIC PRIMER
agaaattatttatagcgatgc

SEQ ID NO: 5 - DNA, 21 bp, Artificial Sequence
  SYNTHETIC PRIMER
attcttccgtgtgtactcagg

SEQ ID NO: 6 - DNA, 21 bp, Artificial Sequence
  SYNTHETIC PRIMER
tggtcaatacggtgatgttca

(19) United States
(12) Patent Application Publication
(10) Pub. No.: US 2024/0109934 A1
(43) Pub. Date: Apr. 4, 2024

(54) ANTIBIOTICS WITH IMPROVED DRUG RESISTANCE PROFILE

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Martin GARCIA CHAVEZ, Urbana, IL (US)

(73) Assignee: THE BOARD OF TRUSTEES OF THE UNIVERSITY OF ILLINOIS, Urbana, IL (US)

(21) Appl. No.: 18/261,373

(22) PCT Filed: Jan. 13, 2022
(86) PCT No.: PCT/US2022/012254

§ 371 Date: Jul. 13, 2023

Related U.S. Application Data
(60) Provisional application No. 63/137,374, filed on Jan. 14, 2021.

(51) Int. Cl. C07J 13/00; A61P 31/04; C07J 41/00

(52) U.S. Cl. CPC C07J 13/007; A61P 31/04; C07J 41/0055

(57) ABSTRACT

Novel Fusidic Acid (FA) based compounds that have equivalent potency against clinical isolates of Staphylococcus aureus (S. aureus) and Enterococcus faecium (E. faecium) as well as an improved resistance profile in vitro when compared to FA. Importantly, the new compounds display efficacy against a FA-resistant strain of Staphylococcus aureus in a soft-tissue murine infection model. This disclosure delineates the structural features of FA necessary for potent antibiotic activity and demonstrates that the resistance profile can be improved for this scaffold and target.

### RELATED APPLICATIONS

This application claims priority under 35 U.S.C. § 119(e) to U.S. Provisional Patent Application No. 63/137,374 filed Jan. 14, 2021, which is incorporated herein by reference.

### GOVERNMENT SUPPORT

This invention was made with government support under Grant No. R01 AI136773 awarded by the National Institutes of Health. The government has certain rights in the invention.

### BACKGROUND OF THE INVENTION

Recent reports have confirmed that the worrisome rise of antibiotic-resistant bacterial infections is continuing. Some of the most problematic Gram-positive pathogens are methicillin-resistant Staphylococcus aureus (MRSA) and vancomycin-resistant Enterococci (VRE), and accordingly have been classified as serious threats by the Centers for Disease Control and Prevention (CDC). The emergence of bacteria resistant to vancomycin, a drug of last resort against Gram-positive infections, highlights the need to develop or re-engineer drugs that could be efficacious against these problematic pathogens.

Fusidic acid is a steroidal antibiotic that is produced by the fungus Fusidium coccineum. Its mechanism of action involves inhibition of protein synthesis by stabilizing the elongation factor G (EF-G) complex, resulting in the truncation of peptide elongation. EF-G being an unusual antibacterial target likely minimizes the cross-resistance of FA with other antibiotics. FA is a Gram-positive-only antibiotic that has been used since the 1960s in Europe to treat Gram-positive infections and has been particularly effective in treating MRSA. To date, FA is approved in 23 countries and is administered via oral, intravenous, and topical formulations. In the United States, FA has achieved its primary and secondary efficacy endpoints in phase 3 clinical trials in the treatment of patients with acute bacterial skin and skin structure infections (ABSSSI). FA has also received Orphan Drug Designation from the FDA for the treatment of prosthetic joint infections, and gained Qualified Infectious Disease Product Designation under the Generating Antibiotic Incentives Now (GAIN) Act. Strikingly, an analysis conducted in 2011 showed that virtually all (99.7%) of S. aureus strains in the United States were susceptible to FA.

Despite the clinical success of FA, the high resistance frequency in the range of 1×10⁻⁶ to 1×10⁻⁸ at 2×-16× the minimum inhibitory concentration (MIC) coupled with drastic MIC shifts upon resistance remain a major concern and are a hindrance to its wider usage. FA resistant bacterial strains display a varied profile, illustrated by the heterogeneity in mutations observed in the laboratory and in the clinic. Some of the most prevalent mutations in the fusA gene (encoding EF-G) observed in the clinic are V90, H457Q, L461K, and A655V. Especially concerning is the L461K mutation, observed clinically in S. aureus strains resistant to FA, which leads to MIC values of ≥256 μg/mL, a >2048-fold increase in MIC upon resistance. This large shift in MIC upon resistance observed from FA from a single amino acid mutation is consistent with the hypothesis that single enzyme inhibitors are more prone to display elevated shifts in MIC upon resistance relative to antibiotics with multiple targets. In addition to mutations in the fusA gene, another source of clinical resistance for FA is the over-expression of the FusB family of proteins encoded by the fusB gene usually carried on a 21-kb plasmid (pUB101); FusB binds to EF-G and drives the dissociation of FA from its target. More recently, fusC and fusD genes have also been discovered in clinical isolates of S. aureus. The fusC gene has been associated with driving resistance in S. aureus and coagulase-negative staphylococci, while fusD gene has been linked to conferring resistance among Staphylococcus saprophyticus.

The aforementioned resistance challenges necessitate a front-loading dosing regimen, with 3 grams of FA taken on the first day of treatment and 1.2 grams daily thereafter, allowing for considerable drug plasma levels to be reached (Cₘₐₓ of greater than 130 μg/mL in patients). Despite multiple efforts to construct FA analogues, none of these derivatives are as active as FA against Gram-positive pathogens; in addition, none of them improve on the resistance issues noted above. No FA derivatives have gained traction translationally, and only FA itself has moved forward to the clinic from this antibiotic class. While no FA derivatives with improved activity against Gram-positive bacteria have been reported, derivatives with improved activity relative to FA have been reported against the malaria parasite Plasmodium falciparum.

Accordingly, there is an urgent need for novel Gram-negative antibiotics and methods of use thereof to treat drug resistant bacteria. There also exists a need for an improved drug resistance profile for compounds that target Gram-negative bacteria.

### SUMMARY

This disclosure provides compounds having improved antibacterial characteristics over the natural product antibiotic fusidic acid. Fusidic acid is used widely as an antibacterial drug outside the US, with over 21 million prescriptions a year. This compound is imperfect as a drug for Gram-positive pathogens due to its high resistance frequency and metabolic liabilities. We have made compounds that have superior properties to fusidic acid for treatment of Gram-positive pathogens. We have also created different compounds that have activity against problematic Gram-negative pathogens.

Accordingly, this disclosure provides a compound of Formula I:

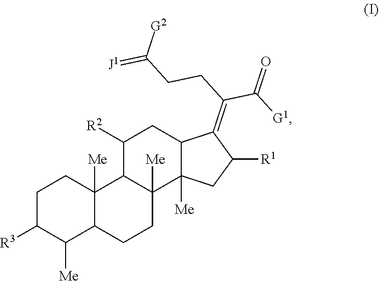

or a salt thereof;
wherein
G¹ is OR^{x}, H, NHOH, NH₂, imidazole, triazole, tetrazole, wherein R^{x} is H or a protecting group;
G² is H, halo, OH, —(C₁-C₆)alkyl, —O(C₁-C₆)alkyl, or NR^{a}R^{b} wherein R^{a} and R^{b} are each independently H, —(C₁-C₆)alkyl, —(C₃-C₆)cycloalkyl, or aryl;
J¹ is CR^{c}R^{d}, O, or absent;
   R^{c} and R^{d} taken together form a cycloalkyl or heterocycloalkyl, wherein a ring bond of cycloalkyl or heterocycloalkyl is optionally an endocyclic double bond; or
   R^{c} and R^{d} are each independently H, halo, aryl, —(C₁-C₆)alkyl, —(C₃-C₆)cycloalkyl, or —(C₀-C₅)R^{e} wherein R^{e} is OH, —O(C₁-C₆)alkyl, or —C(═O)X¹ and X¹ is H, OH, or —O(C₁-C₆)alkyl;
R¹ is —OC(═O)(C₁-C₆)alkyl. H, methyl, ethyl, hydroxy, methoxy, ethoxy, or amino; and
R², and R³ are each independently hydroxy, H, methyl, ethyl, methoxy, ethoxy, amino, or
—OC(═O)(C₁-C₆)alkyl.

This disclosure also provides a method of antimicrobial treatment comprising administering to a subject in need thereof a therapeutically effective amount of a compound described above or a pharmaceutically acceptable salt thereof, thereby killing or inhibiting the growth of at least a portion of a plurality of microorganisms in the subject.

The invention provides novel compounds of Formula I, intermediates for the synthesis of compounds of Formula I, as well as methods of preparing compounds of Formula I. The invention also provides compounds of Formula I that are useful as intermediates for the synthesis of other useful compounds. The invention provides for the use of compounds of Formula I for the manufacture of medicaments useful for the treatment of bacterial infections in a mammal, such as a human.

The invention provides for the use of the compositions described herein for use in medical therapy. The medical therapy can be treating bacterial infections, for example, Gram-negative bacteria. The invention also provides for the use of a composition as described herein for the manufacture of a medicament to treat a disease in a mammal, for example, bacterial infections in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. A). Resistance frequency of FA-CP. B). Resistance frequency of 26. C). Resistance frequency of 16. D). Resistance frequency of 27. Resistance frequency of FA and equipotent analogues was determined in S. aureus ATCC 29213. FA data is the same in all 4 experiments. Error is SEM, n=3. NS=not significant, **p<value 0.01, *** p<value 0.001, and **** p<value 0.0001.

FIG. 2. A). Activity against 29 clinical isolates of S. aureus. B). Activity against six different clinical isolates of E. faecium. MICs determined using CLSI guidelines, n=3. See Table 6 for a list of resistance genes in clinical isolate panels. A full listing of this MIC data is in Tables 7 and 8.

FIG. 3. A). Pharmacokinetic analysis of fusidic acid and FA-CP. C57BL/6 mice were treated with 50 mg/kg of fusidic acid and FA-CP via intraperitoneal injection, with three mice per time point (0, 15, 30, 45, 60, 120, and 240 min). After the stated time points, mice were sacrificed and the serum concentrations of fusidic acid and FA-CP were determined by LC-MS/MS. The data are illustrated as the mean and represent the average of three independent experiments. The pharmacokinetic parameters illustrated above were calculated with a one-compartment model using a non-linear regression program (Phoenix WinNonlin Version 8.1, Certara USA Inc. Princeton NJ 08540 USA). B). S. aureus ATCC 29213 neutropenic thigh infection burden study. CD-1 mice were first rendered neutropenic with cyclophosphamide. Mice were injected on (Day-4 to Day-2) with 150 mg/kg and on (Day-1) with 100 mg/kg of cyclophosphamide, respectively. Acute thigh infections initiated in CD-1 mice with S. aureus ATCC 29213 (8.9×10⁵ c.f.u. mouse⁻¹ via intramuscular injection) were treated with vehicle, fusidic acid, and FA-CP (8 mice per group) at 1 h, 2 h, and 3 h post infection (50 mg/kg via intraperitoneal injection), and the bacterial burden was evaluated at 24 h post-infection. C). S. aureus (FA-32×-B) neutropenic thigh infection study. CD-1 mice were first rendered neutropenic with cyclophosphamide as described above. Acute thigh infections initiated in CD-1 mice with S. aureus (FA-32×-B) (1.08×10⁶ c.f.u. mouse⁻¹ via intramuscular injection) were treated with vehicle, fusidic acid, and FA-CP (8 mice per group) at 1 h, 2 h, and 3 h post-infection (50 mg/kg via intraperitoneal injection), and the bacterial burden was evaluated at 8 h post-infection. Drugs were formulated with 5% DMSO, 10% Tween 20, 85% PBS immediately before injection. The data shown as means±s.d. and statistical significance was determined by one-way ANOVA with Tukey's multiple comparisons. NS indicates not significant (P>0.05), **P<0.01, ****P<0.0001.

### DETAILED DESCRIPTION

Fusidic acid (FA) is a potent steroidal antibiotic that has been used in Europe for more than 40 years to treat a variety of infections caused by Gram-positive pathogens. Despite its clinical success, FA requires significantly elevated dosing (3 grams on the first day, 1.2 grams on subsequent days) to minimize resistance, as FA displays a high resistance frequency and a large shift in minimum inhibitory concentration is observed for resistant bacteria. Despite efforts to improve on these aspects, all previously constructed derivatives of FA have worse antibacterial activity against Gram-positive bacteria than the parent natural product.

Notable challenges in constructing FA variants through direct modification of this natural product include the presence of reactive functional groups such as the olefinic side chain, an α, β-unsaturated carboxylic acid, an acetate, and two sterically hindered alcohols (Scheme 1). Furthermore, the unique chair-boat-chair core conformation of FA makes the total synthesis of this antibiotic challenging and such routes are not yet suitable for the rapid generation of derivatives. Intrigued by the translational potential of FA, here we report the development of synthetic routes from FA that provide rapid access to novel variants with modified side chains, leading to the discovery of a novel FA analogue with an improved resistance profile.

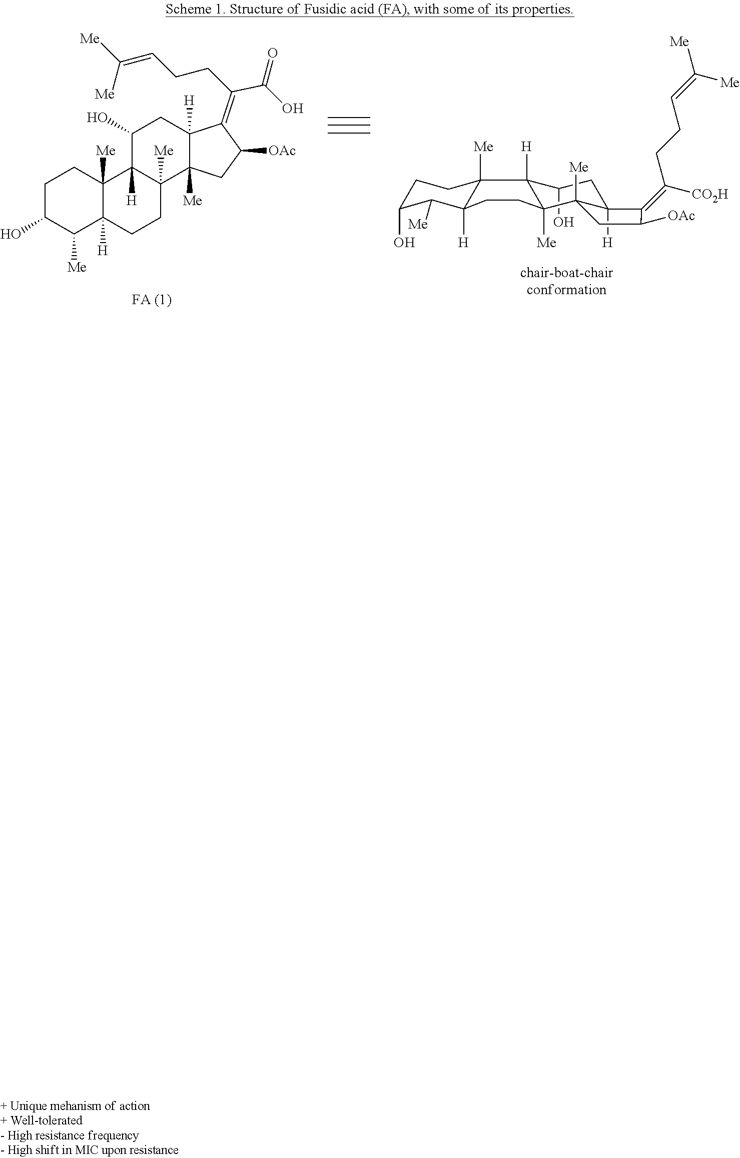

Additional information and data supporting the invention can be found in the following publication by the inventors: ACS Infect. Dis. 2021, 7, 493-505 and its Supporting Information, which is incorporated herein by reference in its entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14′ Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at a carbon atom or at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aromatic” refers to either an aryl or heteroaryl group or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “IC₅₀” is generally defined as the concentration required to kill 50% of the cells in 24 hours.

### Embodiments of the Invention

This disclosure provides a compound of Formula I:

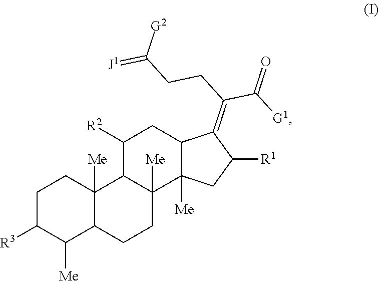

or a salt thereof;
wherein
G¹ is OR^{x}, H, NHOH, NH₂, imidazole, triazole, tetrazole, wherein R^{x} is H or a protecting group;
G² is H, halo, OH, —(C₁-C₆)alkyl, —O(C₁-C₆)alkyl, or NR^{a}R^{b} wherein R^{a} and R^{b} are
each independently H, —(C₁-C₆)alkyl, —(C₃-C₆)cycloalkyl, or aryl;
J¹ is CR^{c}R^{d}, O, or absent;
   R^{c} and R^{d} taken together form a cycloalkyl or heterocycloalkyl, wherein a ring bond of the cycloalkyl or heterocycloalkyl is optionally an endocyclic double bond; or
   R^{c} and R^{d} are each independently H, halo, aryl, —(C₁-C₆)alkyl, —(C₃-C₆)cycloalkyl, or —(C₀-C₅)R^{e} wherein R^{e} is OH, —O(C₁-C₆)alkyl, or —C(═O)X¹ and X¹ is H, OH, or —O(C₁-C₆)alkyl;
R¹ is —OC(═O)(C₁-C₆)alkyl. H, methyl, ethyl, hydroxy, methoxy, ethoxy, or amino; and
R², and R³ are each independently hydroxy, H, methyl, ethyl, methoxy, ethoxy, amino, or —OC(═O)(C₁-C₆)alkyl.

In various embodiments, Formula I is not fusidic acid or a hydrogenated olefin derivative thereof. In other embodiments, G² is not H, halo, alkyl or methyl, OH, alkoxy, NH₂, or aminoalkyl when J¹ is O or absent. In other embodiments, G² is NR^{a}R^{b} when J¹ is absent, or G² is OH or —O(C₁-C₆)alkyl when J¹ is O. In some embodiments, R^{c} and/or R^{d} is not H. In some embodiments, R^{c} and R^{d} are not both H or not both methyl. In various embodiments, R^{c} and R^{d} are each independently —(C₁-C₆)alkyl, —(C₂-C₆)alkyl, —(C₃-C₆)alkyl, —(C₄-C₆)alkyl, —C(═O)O(C₁-C₆)alkyl or —(C₁-C₅)R^{e} wherein R^{e} is defined above. In some embodiments, aryl is substituted with one or more substituents. In other embodiments, the one or more substituents is halo, OH, —(C₁-C₆)alkyl, or —O(C₁-C₆)alkyl.

In some embodiments, the compound is:

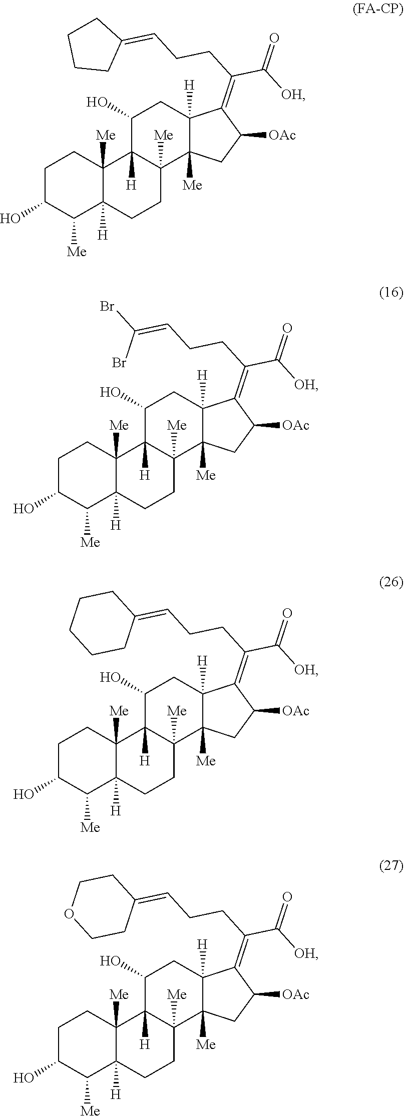

or a salt thereof.

In various embodiments, G¹ is OR^{x}. In various embodiments, OR^{x} is OH. In various embodiments, G² is H. In various embodiments, J¹ is CR^{c}R^{d}. In various embodiments, R^{c} and R^{d} taken together form a cycloalkyl or heterocycloalkyl.

In various embodiments, J¹ is:

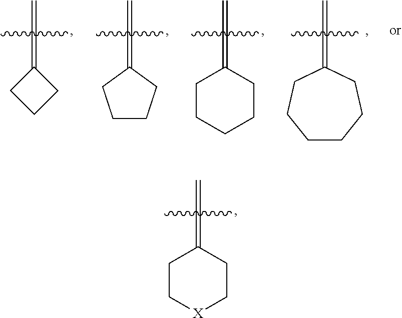

wherein X is O or NR^{f} wherein R^{f} is H, —(C₁-C₆)alkyl, —(C₃-C₆)cycloalkyl, or aryl, and optionally the ring bonds of J¹ comprise at least one endocyclic double bond. In various embodiments, J¹ is:

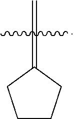 [SMILES: *=C1CCCC1]

In various embodiments, J¹ is:

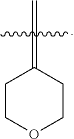 [SMILES: *=C1CCOCC1]

In various embodiments, R^{c} and R^{d} are halo. In various embodiments, halo is bromo. In various embodiments, halo is fluoro, chloro or iodo. In various embodiments, R^{c} is —(C₁-C₆)alkyl and R^{d} is —(C₀-C₅)R^{e}. In various embodiments, R^{d} is —(C₀-C₅)O(C₁-C₆)alkyl. In various embodiments, J¹ is O and G² is —O(C₁-C₆)alkyl. In various embodiments, J¹ is absent and G² is NR^{a}R^{b}.

In various embodiments, the compound has RB of 6 or less, 5 or less, or 4 or less and a Glob of 0.4 or less, 0.3 or less, 0.2 or less, or 0.1 or less; or a combination of any of the foregoing.

In other embodiments, the compound is:

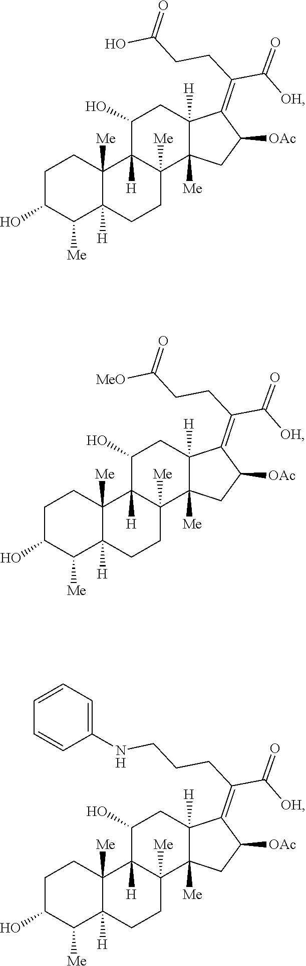

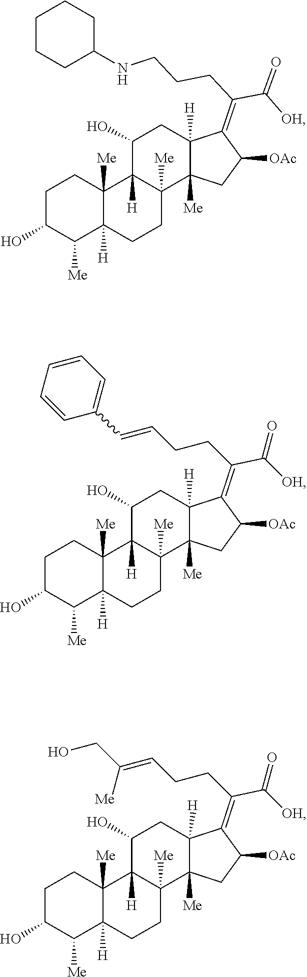

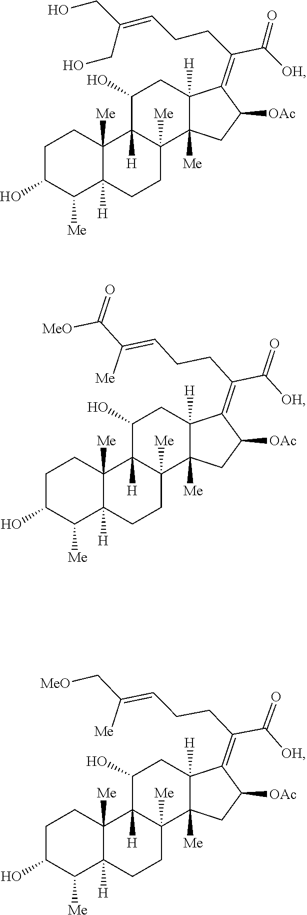

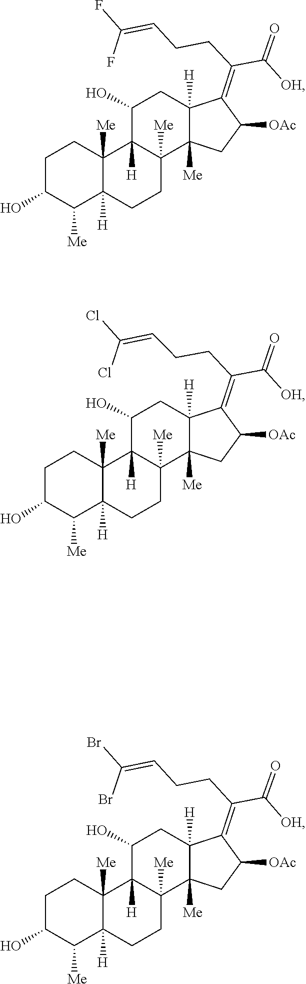

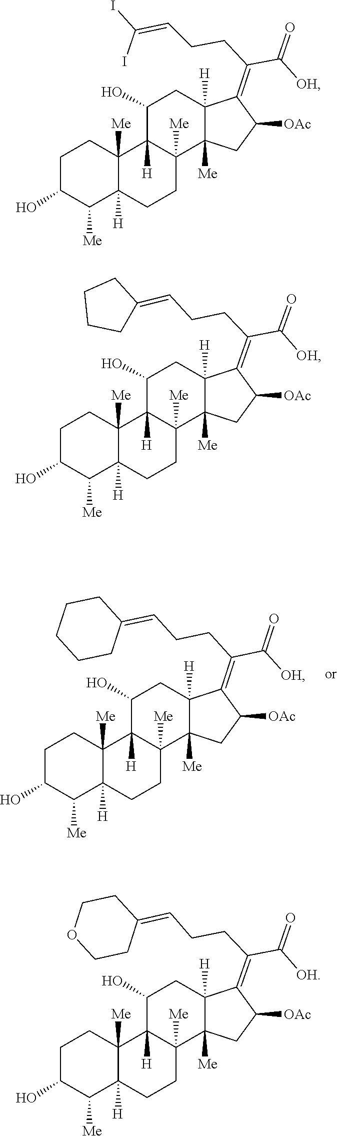

In yet other embodiments, the compound is:

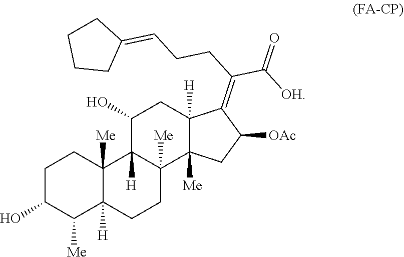

Also, this disclosure provides a pharmaceutical composition comprising a compound disclosed herein and a pharmaceutically acceptable excipient.

Additionally, this disclosure provides a method of antimicrobial treatment comprising administering to a subject in need thereof a therapeutically effective amount of a compound disclosed herein or a pharmaceutically acceptable salt thereof, thereby killing or inhibiting the growth of at least a portion of a plurality of microorganisms in the subject.

In various embodiments, the microorganism is a bacterium. In various embodiments, the microorganism is a Gram-negative bacterium. In other various embodiments, the microorganism is Acinetobacter, anthrax-causing bacteria, Bacilli, Bordetella, Borrelia, botulism-causing bacteria, Brucella, Burkholderia, Campylobacter, Chlamydia, cholera-causing bacteria, Clostridium, Conococcus, Corynebacterium, diptheria-causing bacteria, Enterobacter, Enterococcus, Erwinia, Escherichia, Francisella, Haemophilus, Heliobacter, Klebsiella, Legionella, Leptospira, leptospirosis-causing bacteria, Listeria, Lyme's disease-causing bacteria, meningococcus, Mycobacterium, Mycoplasma, Neisseria, Pasteurella, Pelobacter, plague-causing bacteria, Pneumnonococcus, Proteus, Pseudononas, Rickettsia, Salmonella, Serratia, Shigella, Staphylococcus, Streptococcus, tetanus, Treponema, Vibrio, Yersinia and Xanthomonas, or a combination thereof.

### Other Exemplary Methods

In some embodiments, the compounds disclosed herein accumulate in Gram-negative bacteria or Gram-positive bacteria. In some embodiments, the compounds disclosed herein traverse a porin. In some embodiments, the compound is a compound of Formula (I).

In some embodiments, provided herein is a method of antimicrobial treatment, comprising, providing a sample comprising a plurality of microorganisms; contacting the sample with a compound disclosed herein; thereby killing or inhibiting the growth of at least a portion of the plurality of microorganisms in the sample.

In some embodiments of the methods of antimicrobial treatment disclosed herein, at least a portion of the plurality of microorganisms is killed. In some embodiments of the methods of antimicrobial treatment disclosed herein, the growth of at least a portion of the plurality of microorganisms is inhibited.

In some embodiments, the microorganism is a bacterium, a virus, a fungus, or a parasite. In some embodiments, the microorganism is drug resistant, such as antibiotic resistant. In some embodiments, the microorganism is multi-drug resistant.

In some embodiments, the microorganism is at least one bacterium selected from the group consisting of Acinetobacter baumannii, Escherichia coli, Enterobacter cloacae, Klebsiella pneumoniae, Pseudomonas aeruginosa, and Staphylococcus aureus. In some embodiments, the microorganism is methicillin-resistant Staphylococcus aureus (MRSA). In some embodiments, the microorganism is Pseudomonas aeruginosa.

In some embodiments, for example, the microorganism is at least one virus selected from Adenoviridae, Papillonmaviridae, Polyonaviridae, Herpesviridae, Poxviridae, Hepadnaviridae, Parvoviridae, Astroviridae, Caliciviridae, Picornaviridae, Coronoviridae, Flaviviridae, Retroviridae, Togaviridae, Arenaviridae, Bunyaviridae, Filoviridae, Orthonyxoviridae, Paramyxoviridae, Rhabdoviridae, and Reoviridae. In certain embodiments, the virus may be arboviral encephalitis virus, adenovirus, herpes simplex type I, herpes simplex type 2, Varicella-zoster virus, Epstein-barr virus, cytomegalovirus, herpesvirus type 8, papillomavirus, BK virus, coronavirus, echovirus. JC virus, smallpox, Hepatitis B3, bocavirus, parvovirus B19, astrovirus, Norwalk virus, coxsackievirus, Hepatitis A, poliovirus, rhinovirus, severe acute respiratory syndrome virus, Hepatitis C, yellow fever, dengue virus, West Nile virus, rubella, Hepatitis F, human immunodeficiency virus (HIV), human r-cell lymphotropic virus (HTLV), influenza, guanarito virus, Junin virus, Lassa virus, Machupo virus, Sabia virus, Crimean-Congo hemorrhagic fever virus, ebola virus, Marburg virus, measles virus, molluscum virus, mumps virus, parainfluenza, respiratory syncytial virus, human metapneumovirus, Hendra virus, Nipah virus, rabies, Hepatitis D, rotavirus, orbivirus, coltivirus, vaccinia virus, and Banna virus.

In some embodiments, for example, the microorganism is at least one fungus selected from Aspergillus (fumigatus, niger, etc.), Basidiobolus (ranarum, etc.), Blastomyces dermatitidis, Candida (albicans, krusei, glabrata, tropicalis, etc.), Coccidioides immitis, Cryptococcus (neoformans, etc.), eumycetoma, Epidermophyton (floccosum, etc.), Histoplasma capsulatum, Hortaea werneckii, Lacazia loboi, Microsproum (audouinii, nanum etc.), Mucorales (mucor, absidia, rhizophus), Paracoccidioides brasiliensis, Rhinosporidium seeberi, Sporothrix schenkii, and Trichophyton (schoeleinii, mentagrophytes, rubrum, verrucosum, etc.).

In some embodiments, for example, the microorganism is at least one parasite selected from Acanthanoeboa, Babesia microti, Balantidium coli, Entamoeba hystolylica, Giardia lamblia, Cryptosporidium muris, Trypanosomatida gambiense, Trypanosomatida rhodesiense, Trypanosoma brucei, Trypanosomna cruzi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Pneumocystis carinii, Trichomonas vaginalis, Histomonas meleagridis, Secementea, Trichuris trichiura, Ascaris lumbricoides, Enterobius vermicularis, Ancylostoma duodenale, Naegleria fowleri, Necator americanus, Nippostrongylus brasiliensis, Strongyloides stercoralis, Wuchereria bancrofti, Dracunculus medinensis, blood flukes, liver flukes, intestinal flukes, lung flukes, Schistosoma mansoni, Schistosoma haematobium, Schistosoma japonicum, Fasciola hepatica, Fasciola gigantica, Heterophyes heterophyes, and Paragonimus westermani.

### Results

Synthesis and antibacterial assessment of FA derivatives where the carboxylic acid and alcohols are modified. Key to this study was to first determine the important structural features necessary to retain the potent antibacterial activity of FA. Although FA derivatives have been reported in the literature, a systematic antimicrobial activity assessment of FA derivatives with MIC values against key Gram-positive pathogens does not exist. Thus, as a prelude to our work, we first synthesized several known FA analogues and assessed their antibacterial activity, with the specific goals of probing the importance of the carboxylic acid and alcohols.

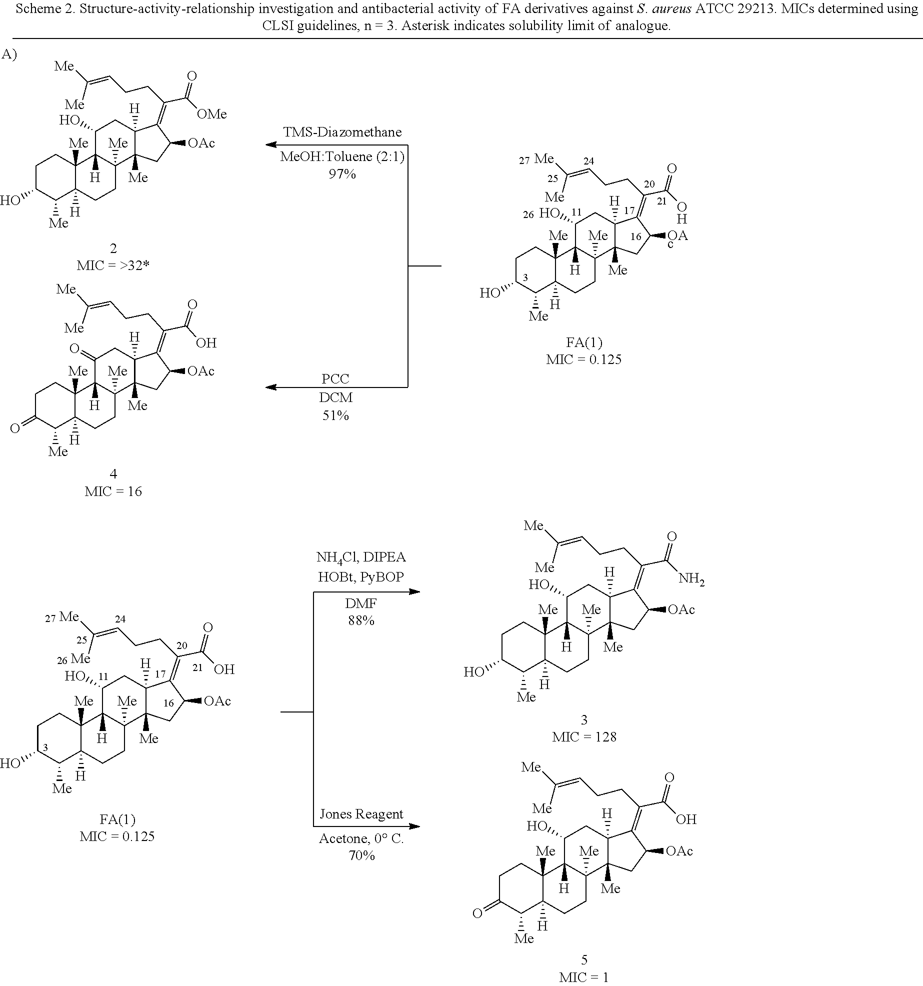

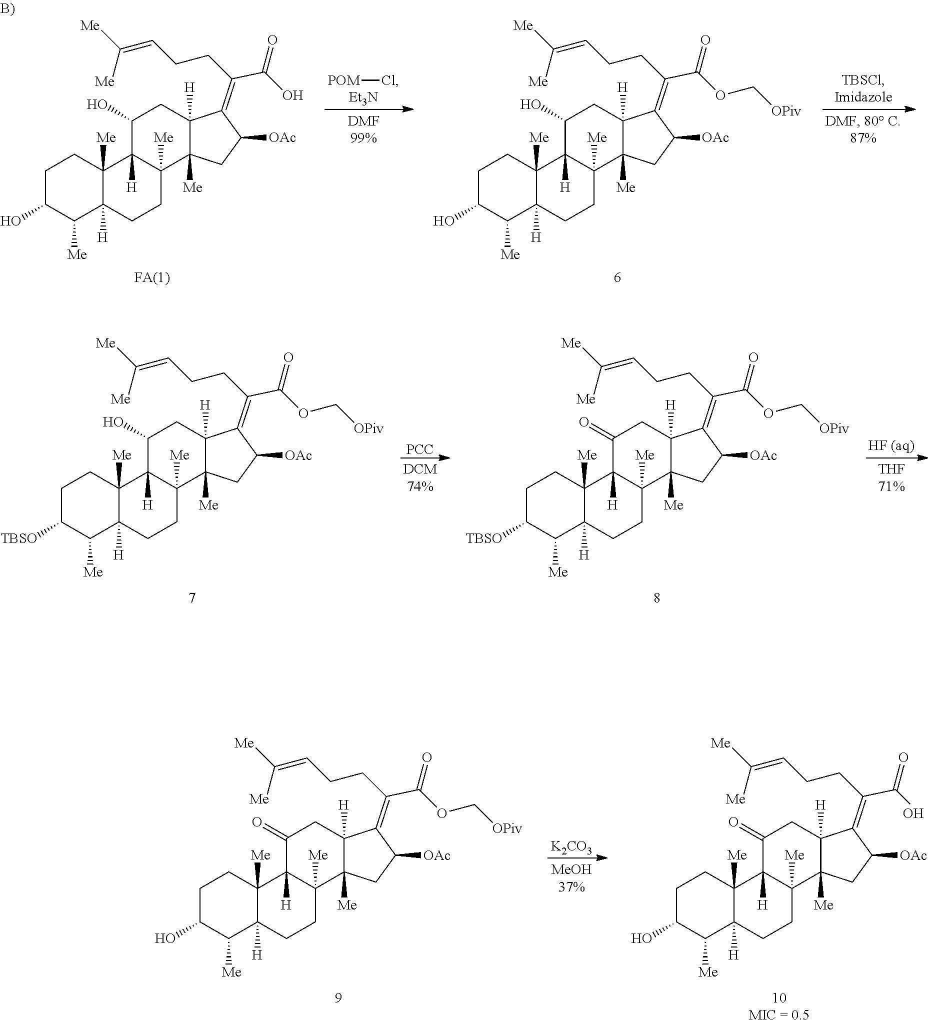

The role of the carboxylic acid was investigated via construction of ester 2 and amide 3, using previously published routes (Scheme 2A) (Medchemcomm 2015, 6 (11), 2023; Nat. Chem. 2017, 9 (12), 1213). Diketone 4 was synthesized previously using the Jones oxidation reaction. Herein, diketone 4 was synthesized using pyridinium chlorochromate (Scheme 2A). Oxidation solely at C3 was effected using recently published conditions to provide compound 5 (Scheme 2A) (ACS Infect. Dis. 2019, 5 (9), 1634). The mono-ketone at C11 was previously synthesized by treating FA with Kiliani's reagent in a low yielding process. Herein, a five-step sequence was developed to generate the C11 ketone (Scheme 2B). This route required the protection of the carboxylic acid to generate intermediate 6. The steric hindrance at the C11 position induced by 1,3-diaxial interactions was leveraged, and the C3 alcohol was selectively protected with TBS to generate 7. The C11 alcohol was then oxidized with pyridinium chlorochromate to generate 8. Removal of the TBS protecting group in compound 8 with aqueous hydrofluoric acid provided 9, and subsequent deprotection of the pivaloyloxymethyl protecting group under mild basic conditions afforded the target mono-ketone, compound 10 (Scheme 2B). Key to this work was utilizing a suitable protecting group for the carboxylic acid that could be removed under mild basic conditions as lactonization between the C16 acetyl and the C21 carboxylic acid occurs under strongly basic conditions. Accordingly, chloromethyl pivalate, used previously to protect the FA carboxylic acid, was selected based on its ability to be removed under mild conditions.

Antibacterial activity of FA analogues 2-5 and 10 was assessed against S. aureus ATCC 29213 using standard MIC assays following Clinical and Laboratory Standards Institute (CLSI) guidelines. As shown in Scheme 2, FA analogues 2-4 display significantly reduced antibacterial activity, reinforcing the importance of the free carboxylic acid and both of the alcohols. Oxidation of the secondary alcohols separately is more tolerated, with the mono-ketones at C3 (compound 5) and at C11 (compound 10) having MIC values only 4-8-fold worse than FA (Scheme 2).

Synthesis and antibacterial evaluation of side chain analogues. As modifications to FA shown in Scheme 2 were all deleterious to antibacterial activity, the focus shifted to modifications of the gem-dimethyl alkene side chain. This work was informed by the crystal structure of the ribosome trapped with EF-G in a post-translocational state with FA bound; this complex indicates that the side chain of FA is positioned in a large, hydrophobic pocket of EF-G.

Several FA analogues with modifications to the side chain have been previously reported such as derivatives where the C24 and C25 or the C17 and C20 double bonds have been reduced. Furthermore, truncation of the C26 and C27 methyl groups has also been reported as well as analogues where the C26 and C27 methyl groups where modified via microbial oxidation. Heteroatoms have also been introduced via ozonolysis of the C24 and C25 double bond. Aryl side chain analogues of FA have were also synthesized and used to conduct photolabeling studies. Notably, all of the previously reported side chain analogues of FA have reduced antibacterial activity relative to FA.

To provide access to FA derivatives with novel side chains, a modular synthetic route for rapid generation of a variety of compounds with modified side chains was envisioned. Aldehyde 11, generated previously by ozonolysis and herein via dihydroxylation followed by oxidative cleavage (Scheme 3A), was selected as the pluripotent intermediate that could be transformed via olefinations, one-carbon homologations, and cross-metathesis reactions to the various analogues. Using 11, halogens were introduced via a one-carbon homologation reaction with carbon tetrachloride, carbon tetrabromide, and carbon tetraiodide, respectively, to generate intermediates 12-14 (Scheme 3B). Subsequent deprotection with potassium carbonate in methanol afforded novel halogenated compounds 15, 16, and 17 (Scheme 3B). Construction of the analogous fluorinated analogue was accomplished by treating aldehyde 11 with chlorodifluoroacetic acid and triphenylphosphine at elevated temperatures to generate 18 and deprotection to generate difluorinated derivative 19 (Scheme 3C). Aldehyde 11 was also converted to olefin 20 and fully deprotected to generate 21 as previously reported (Scheme 3D) (CN105924488).

Olefin 20 was used as another key point of diversity and divergence to generate compounds containing symmetrical side chains with the C24 and C25 double bond intact. Thus, to form cyclic derivatives, olefin 20 was coupled via a cross metathesis reaction with methylenecyclopentane, methylenecyclohexane, and 4-methylenetetrahydropyran using Grubbs Catalyst 2 to generate analogues 22-24. Deprotection generated novel cyclic side chain derivatives 25, 26, and 27 (Scheme 3E). Lastly, compound 28 with a fully saturated sidechain was synthesized as previously reported using Lindlar's catalyst (Scheme 3F).

With this collection of FA derivatives in hand, the MIC of each compound against S. aureus was determined (Scheme 3). Compounds 15, 17, 19, 21, and 28 displayed modest decreases in potency relative to FA. Notably, analogues 16, 25 (hereafter referred to as FA-CP), 26, and 27 displayed equipotent activity to FA, suggesting the antibiotic potential of FA derivatives with modified sidechains (Scheme 3).

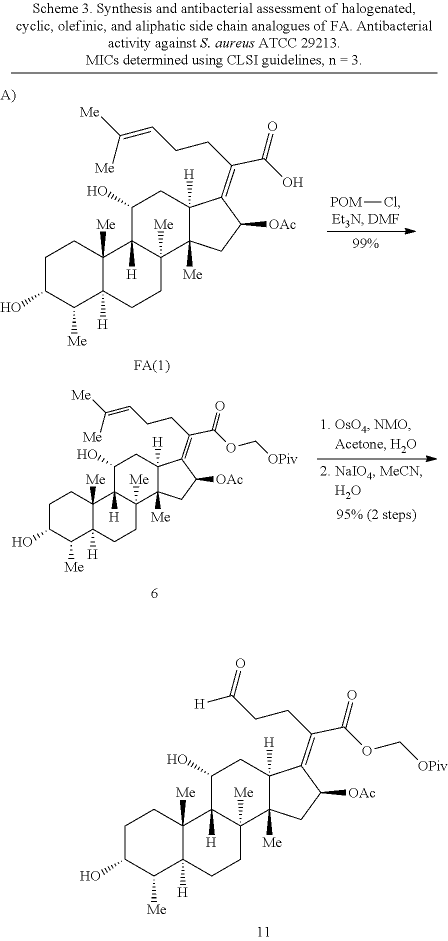

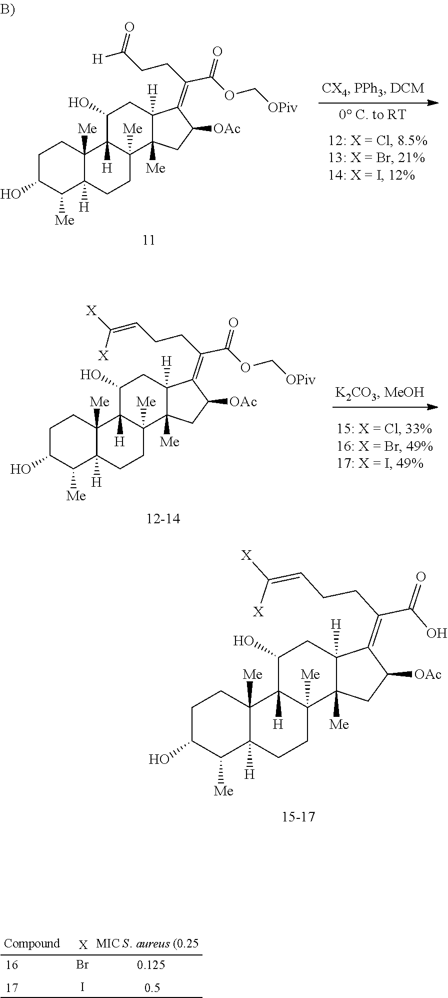

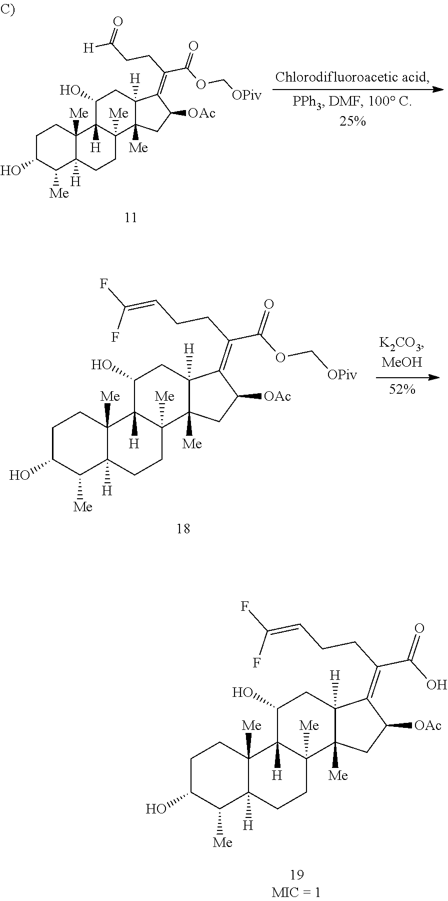

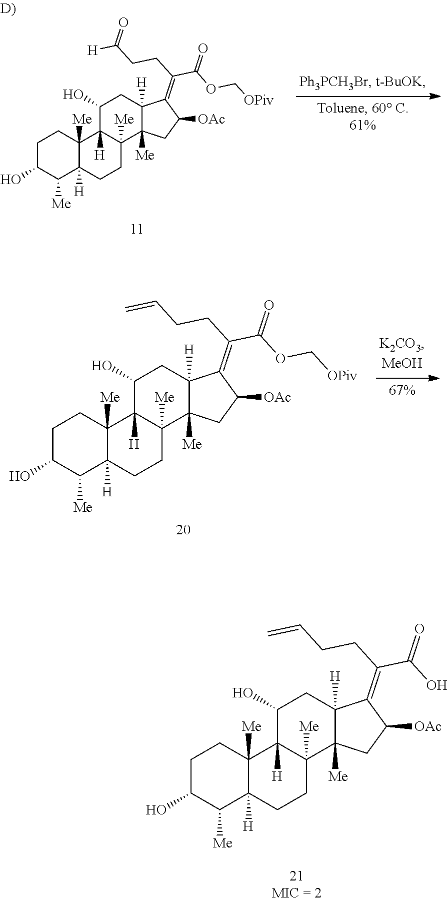

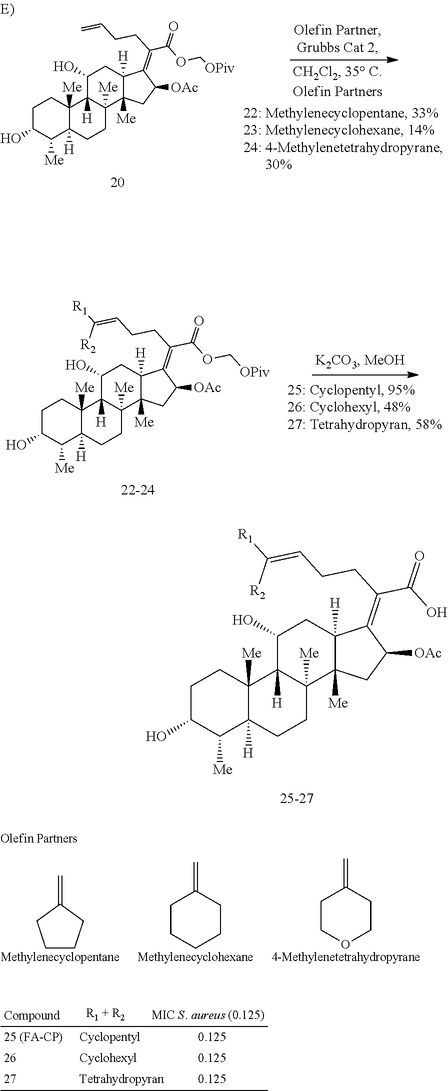

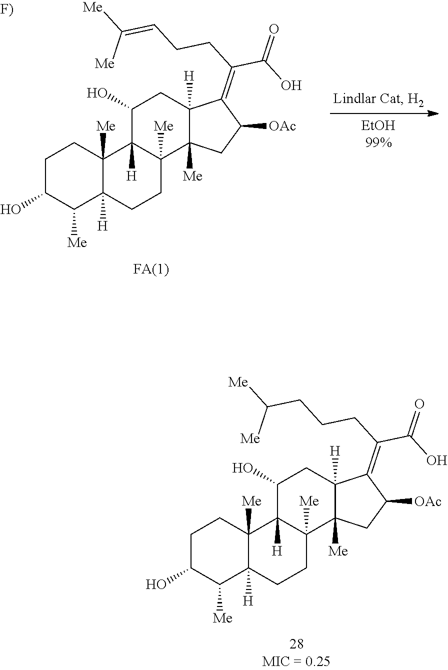

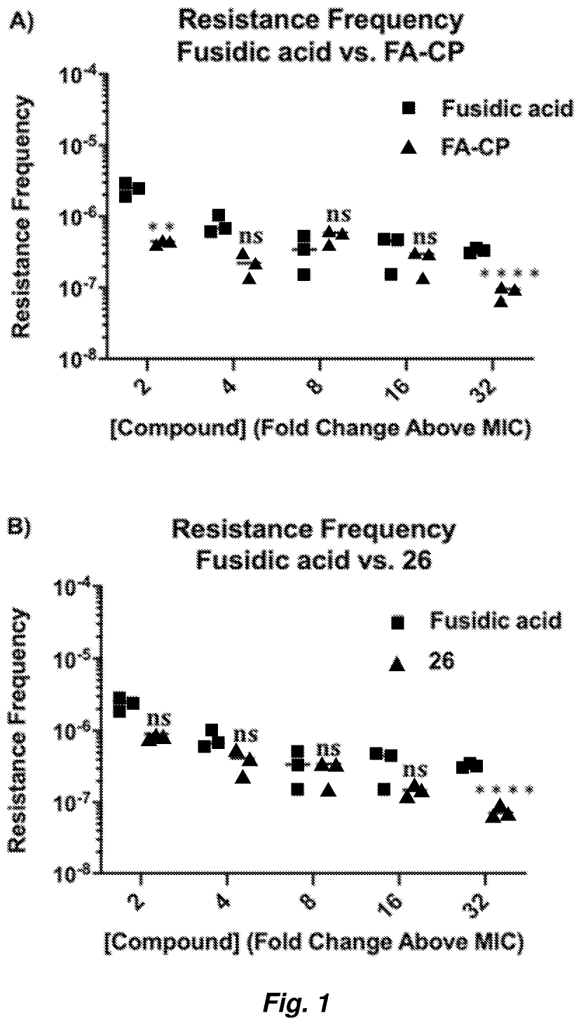

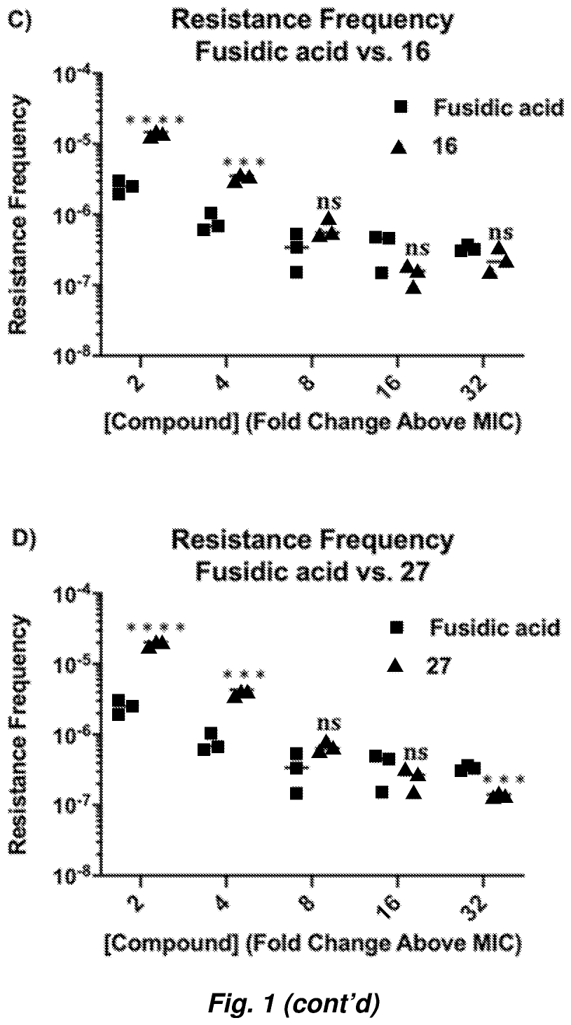

Evaluation of resistance frequency of equipotent analogues of FA. The resistance frequency of the equipotent analogues (16, 25 (FA-CP), 26, and 27) was assessed in head-to-head experiments with FA. The large inoculum method was used in order to generate resistance mutants and derive the resistance frequency of FA and the equipotent analogues. The resistance frequency was determined at 2×, 4×, 8×, 16×, and 32× the MIC, which is 0.125 μg/mL for all compounds. The resistance frequency at the listed MICs was then compared head-to-head against FA and statistical significance was then determined using one-way ANOVA with Tukey's multiple comparisons. As shown in FIGS. 1A and 1B, compounds FA-CP and 26 displayed an improvement in resistance frequency relative to FA. FA-CP displayed a modest improvement in resistance frequency at 2× and 32× the MIC, while analogue 26 only displayed an improvement at 32× the MIC (FIG. 1A and FIG. 1B). This is in contrast with analogues 16 and 27, which displayed a worse resistance frequency relative to FA (FIG. 1C and FIG. 1D).

Effect of human serum binding, assessment of metabolic stability, mammalian cell toxicity. The translational potential of the novel derivatives was further investigated by assessing parameters important in antibacterial development, such as the effect of human serum binding, metabolic stability, and mammalian cell toxicity. FA is reported to be highly protein bound, with human plasma protein binding of 97%. Additionally, the MIC for FA when 50% human serum is included in the media shifts to 16-256× higher. In this work, the shift in MIC for S. aureus in 50% human serum was determined to be very similar for FA as compared to compounds 16, FA-CP, 26, 27 (Table 1). In addition, the stability of the novel derivatives to mouse liver microsomes was similar to FA (Table 2). FA, 16, FA-CP, 26, 27 exhibited low toxicity against human foreskin fibroblast-1 cells (HFF-1), with all compounds giving IC₅₀ values greater than 50 μM (Table 3).

TABLE 1
Structures of FA and compounds 16, FA-CP, 26, and 27. MIC's of FA and
16, FA-CP, 26, 27 in S. aureus 29213 without human serum and fold changes
increase in MIC of FA, 16, FA-CP, 26, 27 in S. aureus 29213 with 50% human serum.

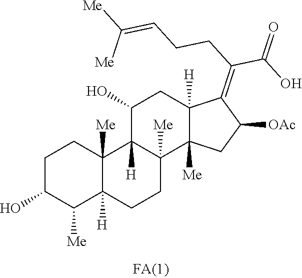

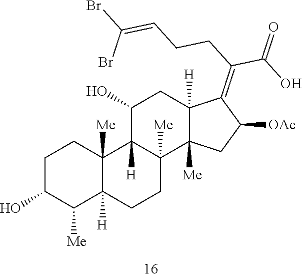

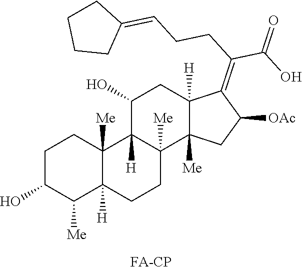

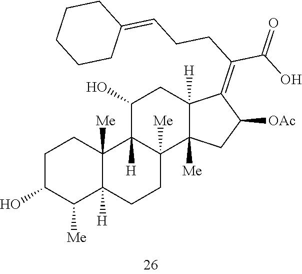

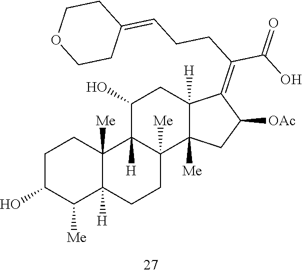

| | MIC (μg/mL) Without Human Serum | | | | |
|---|---|---|---|---|---|
| WT Strain | FA | 16 | FA-CP | 26 | 27 |
| S. aureus 29123 | 0.125 | 0.125 | 0.125 | 0.125 | 0.125 |
| | Fold Change Increase in MIC with 50% Human Serum | | | | |
| WT Strain | FA | 16 | FA-CP | 26 | 27 |
| S. aureus 29123 | 16X | 32X | 8X | 16X | 32X |

MICs in μg/mL, (n = 3).

TABLE 2
Structures of FA and compounds 16, FA-CP, 26, and 27. Metabolic
stability of FA, 16, FA-CP, 26, and 27 after incubation
for 3 h at 37° C. in mouse liver microsomes. Percent
remaining was quantified using LC-MS/MS. Error =
standard error of the mean of 3 biological replicates.

| | Metabolic Stability |
|---|---|
| Compound | in Mouse Liver Microsomes |
| FA(1) | 82 ± 0.8% |
| 16 | 88 ± 2% |
| FA-CP | 78 ± 4% |
| 26 | 61 ± 1% |
| 27 | 86 ± 5% |

TABLE 3
Structures of FA and compounds 16, FA-CP, 26, and 27. IC₅₀ values
(μM) in human foreskin fibroblasts (HFF-1) of FA, 16, FA-CP,
26, and 27. Cells were incubated for 72 h before viability was
assessed by the Alamar Blue Assay. Error is SEM, n = 2.

| Compound | Mammalian Toxicity in HFF-1 | |
|---|---|---|
| FA(1) | 146 ± 11 | μM |
| 16 | 75 ± 3 | μM |
| FA-CP | 112 ± 2 | μM |
| 26 | 53 ± 4 | μM |
| 27 | 409 ± 16 | μM |

Mode-of-action of novel FA derivative. FA-CP was selected for additional studies investigating the mode-of-action. For these studies, resistance mutants were generated at 32× the MIC of FA-CP and FA, and sequencing of the fusA gene (encoding for EF-G, the target of FA) revealed mutations in fusA, suggesting that FA-CP engages the same target as FA. Specifically, for FA and FA-CP, the fusA gene was sequenced for 40 different resistant colonies generated at 32× the MIC, 20 colonies for each compound. As shown in Table 4, the resistance mutants observed for FA are in accord with those seen previously in analogous studies. Specifically, mutations F88L, T436I, H457N, and D434N have been identified in bacterial cell culture studies with S. aureus and FA. Additionally, H457Y has been observed in bacterial cell culture studies as well as clinical isolates of S. aureus that are resistant to FA. Consistent with the notion that FA-CP engages the same target as FA, single amino acid substitutions (H457Y, D434N, and F88L) within EF-G were found in the 20 different FA-CP colonies (Table 4).

The MICs of FA and FA-CP against these resistance mutants were also determined, and it was found that the mutants generated from FA-CP typically displayed a reduced shift in MIC upon resistance relative to the mutants generated from FA (Table 4). Specifically, the highest MIC observed for colonies generated from FA-CP was 64 μg/mL (Fold increase from WT S. aureus: 512) (Table 4). In our in-house studies, the highest MIC observed for FA was 256 μg/mL (Fold increase from WT S. aureus: 2048) (Table 4).

TABLE 4
Observed mutations in the EF-G protein (encoded by fusA) and
MIC values for FA and FA-CP at 32X the MIC in S. aureus.

| Strains | | MIC | Strains | | MIC |
|---|---|---|---|---|---|
| Resistant | EF-G | for | Resistant | EF-G | for |
| to FA | Mutation | FA | to FA-CP | Mutation | FA-CP |
| FA-32X-1 | H457Y | 128 | FA-CP-32X-1 | H457Y | 32 |
| FA-32X-2 | T436I | 8 | FA-CP-32X-2 | H457Y | 64 |
| FA-32X-3 | H457Y | 128 | FA-CP-32X-3 | H457Y | 64 |
| FA-32X-4 | H457N | 256 | FA-CP-32X-4 | H457Y | 32 |
| FA-32X-5 | F88L | 256 | FA-CP-32X-5 | H457Y | 64 |
| FA-32X-6 | T436I | 16 | FA-CP-32X-6 | H457Y | 64 |
| FA-32X-7 | H457Y | 128 | FA-CP-32X-7 | H457Y | 64 |
| FA-32X-8 | H457Y | 128 | FA-CP-32X-8 | H457Y | 64 |
| FA-32X-9 | F88L | 256 | FA-CP-32X-9 | H457Y | 32 |
| FA-32X-10 | H457Y | 128 | FA-CP-32X-10 | D434N | 32 |
| FA-32X-11 | F88L | 256 | FA-CP-32X-11 | H457Y | 32 |
| FA-32X-12 | H457Y | 128 | FA-CP-32X-12 | D434N | 32 |
| FA-32X-13 | H457Y | 128 | FA-CP-32X-13 | H457Y | 64 |
| FA-32X-14 | H457Y | 128 | FA-CP-32X-14 | H457Y | 32 |
| FA-32X-15 | H457Y | 128 | FA-CP-32X-15 | H457Y | 64 |
| FA-32X-16 | F88L | 256 | FA-CP-32X-16 | F88L | 64 |
| FA-32X-17 | H457N | 256 | FA-CP-32X-17 | F88L | 64 |
| FA-32X-18 | D434N | 256 | FA-CP-32X-18 | H457Y | 64 |
| FA-32X-19 | H457Y | 128 | FA-CP-32X-19 | H457Y | 32 |
| FA-32X-20 | H457Y | 128 | FA-CP-32X-20 | H457Y | 32 |

In a separate experiment, S. aureus strains resistant to FA were generated at 4×, 8×, 16×, and 32× the MIC. Mutations found in the fusA gene of the resistant colonies are consistent with what has been observed in previous studies conducted with FA. Specifically, observed mutations in the fusA gene causing single the single amino acid substitutions T385N, R464L, F88L, H457L, T436I, H457N, and D434N have been identified in bacterial cell culture studies with S. aureus and FA. Furthermore, H457Y and P406L have been observed in bacterial cell culture studies as well as clinical isolates of S. aureus that are resistant to FA. FA-CP was evaluated against these eleven FA-resistant strains for antimicrobial activity. Although FA-CP displays cross-resistance with FA, expected as the target is conserved between the two compounds, FA-CP displays a 2-8-fold improvement in activity against these FA-resistant strains relative to FA (Table 5).

TABLE 5
MIC values for FA and FA-CP against 11 different FA-Resistant
strains of S. aureus. FA-resistant strains were generated
at 4x (n = 2), 8x (n = 2), 16x (n = 1),
or 32x (n = 6) the MIC as indicated. MICs determined
using CLSI guidelines, n = 3.

| Strains | EF-G | MIC | MIC |
|---|---|---|---|
| Resistant to FA | Mutation | for FA | for FA-CP |
| FA-4X-A | T385N | 8 | 2 |
| FA-4X-B | T385N | 8 | 2 |
| FA-8X-A | T385N | 8 | 2 |
| FA-8X-B | R464L | 16 | 8 |
| FA-16X-A | H457Y | 128 | 64 |
| FA-32X-A | F88L | 256 | 128 |
| FA-32X-B | P406L | 32 | 4 |
| FA-32X-C | H457L | 256 | 128 |
| FA-32X-D | T436I | 8 | 2 |
| FA-32X-E | H457N | 256 | 128 |
| FA-32X-F | D434N | 256 | 32 |

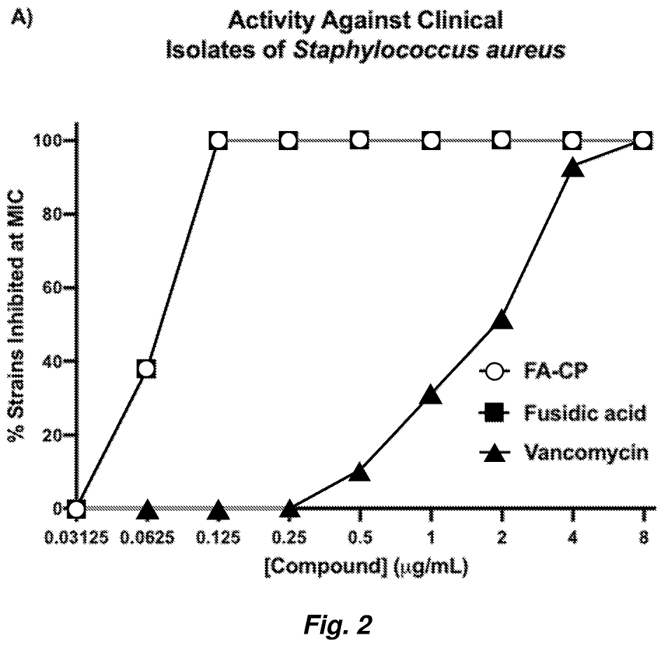

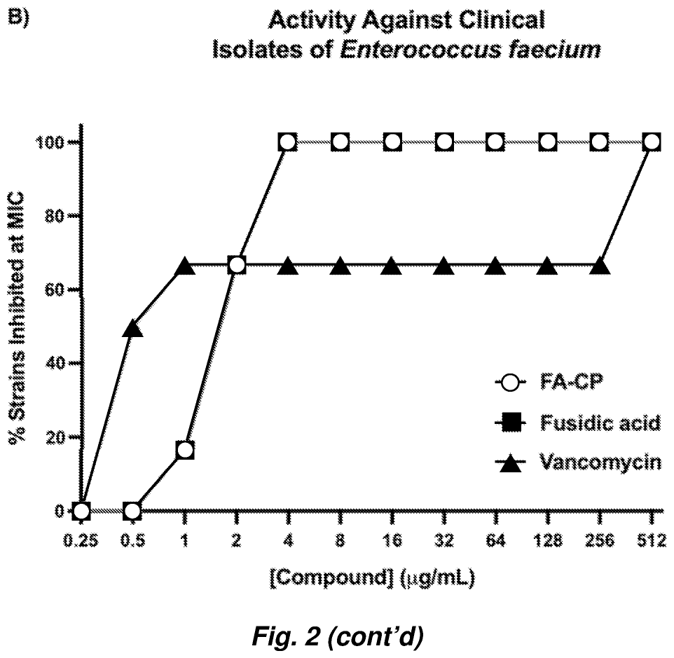

Activity of FA-CP and FA against multidrug-resistant clinical isolates of S. aureus, E. faecium, and a S. aureus strain harboring the 31usc gene. The activity of FA, FA-CP, and vancomycin was assessed against 29 different clinical isolates of S. aureus. FA and FA-CP displayed no cross-resistance with vancomycin and retain potent activity against the 29 different clinical isolates assessed (FIG. 2A). Noteworthy is that within this collection of clinical isolates there are strains with resistance to a wide variety of antibiotics, including cefoxitin, clindamycin, doxycycline, erythromycin, gentamicin, levofloxacin, oxacillin, penicillin, tetracycline, and trimethoprim/sulfamethoxazole (see Table 6 and 7 for a full list of resistance profiles).

TABLE 6
List of antibiotics and associated reference numbers with clinically
relevant levels of resistance in S. aureus and E. faecium
clinical isolates assessed in Table 7 and Table 8.

| Reference No. | Antibiotic |
|---|---|
| 1 | Cefoxitin |
| 2 | Ampicillin |
| 3 | Clindamycin |
| 4 | High-level Gentamicin |
| 5 | Doxycycline |
| 6 | Erythromycin |
| 7 | Gentamicin |
| 8 | Levofloxacin |
| 9 | High-level Streptomycin |
| 10 | Mupirocin |
| 11 | Oxacillin |
| 12 | Penicillin |
| 13 | Rifampin |
| 14 | Quinupristin/dalfopristin |
| 15 | Tetracycline |
| 16 | Trimethoprim/sulfamethoxazole |
| 17 | Vancomycin |
| 18 | Teicoplanin |
| 19 | Ciprofloxacin |
| | — |

TABLE 7
Antimicrobial assessment of FA, vancomycin, and FA-CP in panel of
multidrug-resistant clinical isolates of S. aureus. This data
is depicted in FIG. 2A. Full list of antibiotic resistance is provided.
MICs determined using CLSI guidelines and are listed in μg/mL.
All experiments were performed in biological triplicate.

| | Drug | Vancomycin | FA | FA-CP |
|---|---|---|---|---|
| Strain | Resistance | MIC | MIC | MIC |
| S. aureus AR0561^{a} | 1, 8, 10-12, 15 | 1 | 0.125 | 0.125 |
| S. aureus AR0562^{a} | 1, 3, 6, 8, | 2 | 0.125 | 0.125 |
| | 11-12, 15 | | | |
| S. aureus AR0563^{a} | 1, 6-7, 10-12 | 1 | 0.125 | 0.125 |
| S. aureus AR0564^{a} | 1, 3, 6-8, 10-12 | 2 | 0.125 | 0.125 |
| S. aureus AR0565^{a} | 1, 3, 5-8, | 2 | 0.0625 | 0.0625 |
| | 11-12, 15-16 | | | |
| S. aureus AR0566^{a} | 1, 7-8, 11-12 | 0.5 | 0.125 | 0.125 |
| S. aureus AR0567^{a} | 1, 3, 6-8, | 1 | 0.125 | 0.125 |
| | 10-12, 17 | | | |
| S. aureus AR0568^{a} | 3, 6, 8, 12 | 0.5 | 0.125 | 0.125 |
| S. aureus AR0570^{a} | 1, 3, 6-8, | 1 | 0.125 | 0.125 |
| | 10-12, 15 | | | |
| S. aureus NRS3^{b} | 7, 11-12, 19 | 4 | 0.0625 | 0.0625 |
| S. aureus NRS4^{b} | 11-12, 19 | 4 | 0.0625 | 0.0625 |
| S. aureus NRS17^{b} | 11-12, 19 | 8 | 0.0625 | 0.0625 |
| S. aureus NRS18^{b} | 11-12, 19 | 4 | 0.125 | 0.125 |
| S. aureus NRS21^{b} | 7, 11-12, 15, | 4 | 0.0625 | 0.0625 |
| | 16, 19 | | | |
| S. aureus NRS22^{b} | 3, 6-7, 11-12, | 4 | 0.0625 | 0.0625 |
| | 16, 19 | | | |
| S. aureus NRS23^{b} | 11-12, 16, 19 | 4 | 0.125 | 0.125 |
| S. aureus NRS24^{b} | 7, 11-12, 19 | 4 | 0.0625 | 0.0625 |
| S. aureus NRS26^{b} | 7, 11-12, | 2 | 0.0625 | 0.0625 |
| | 16, 19 | | | |
| S. aureus NRS27^{b} | 7, 11-12, | 4 | 0.125 | 0.125 |
| | 16, 19 | | | |
| S. aureus NRS29^{b} | 7, 11-12, | 2 | 0.125 | 0.125 |
| | 16, 19 | | | |
| S. aureus NRS51^{b} | 11-12, 19 | 4 | 0.0625 | 0.0625 |
| S. aureus NRS68^{b} | 3, 6, 11-12, 19 | 4 | 0.125 | 0.125 |
| S. aureus NRS73^{b} | 7, 11-12, 16, | 4 | 0.125 | 0.125 |
| | 18 19 | | | |
| S. aureus NRS74^{b} | 3, 6-7, 11-12, 19 | 8 | 0.125 | 0.125 |
| S. aureus NRS76^{b} | 6, 11-12, 19 | 4 | 0.125 | 0.125 |
| S. aureus NRS77^{b} | No noted | 1 | 0.0625 | 0.0625 |
| | resistance | | | |
| S. aureus NRS382^{b} | 3, 6, 11-12, 19 | 2 | 0.0625 | 0.0625 |
| S. aureus NRS383^{b} | 3, 6-7, 11-12, 19 | 0.5 | 0.125 | 0.125 |
| S. aureus NRS384^{b} | 6, 11-12 | 1 | 0.125 | 0.125 |

^{a}Obtained from the Centers for Disease Control and Prevention (CDC).
^{b}Obtained from the Network on Antimicrobial Resistance in Staphylococcus aureus (NARSA).

The activity of FA and FA-CP was also assessed against a S. aureus strain that possesses the fusC gene, strain S. aureus ATCC BAA-1721. FA-CP displayed cross-resistance with FA as both compounds displayed an MIC of 8 μg/mL against this strain.

The activity of FA, FA-CP, and vancomycin was also assessed against six different clinical isolates of E. faecium (FIG. 2B). As a reference point, the MIC of FA and FA-CP against a sensitive strain of E. faecium ATCC 19434 is 2 μg/mL for both antibacterial agents. FA and FA-CP displayed no cross-resistance with vancomycin in the E. faecium clinical isolates. Excitingly, FA and FA-CP retained potent activity against VRE with high levels of vancomycin resistance (Vancomycin MIC=512 μg/mL). See Table 8 for a full list of resistance profiles.

TABLE 8
Antimicrobial assessment of FA, vancomycin, and FA-CP in panel of
multidrug-resistant clinical isolates of E. faecium. This data
is depicted in FIG. 2B. Full list of antibiotic resistance is provided.
MICs determined using CLSI guidelines and are listed in μg/mL.
All experiments were performed in biological triplicate.

| | Drug | Vancomycin | FA | FA-CP |
|---|---|---|---|---|
| Strain | Resistance | MIC | MIC | MIC |
| E. faecium AR0572^{a} | 2, 5, 8, 12, | 512 | 4 | 4 |
| | 13, 17-18 | | | |
| E. faecium AR0574^{a} | 2, 5, 8-9, 12, 13 | 0.5 | 2 | 2 |
| E. faecium AR0575^{a} | 2, 8, 12, 13, 17-18 | 512 | 2 | 2 |
| E. faecium AR0576^{a} | 2, 5, 8, 12-13 | 1 | 4 | 4 |
| E. faecium AR0578^{a} | 2, 4-5, 8, 12, 13 | 0.5 | 2 | 2 |
| E. faecium AR0579^{a} | 2, 4-5, 9, 12, 13-14 | 0.5 | 1 | 1 |

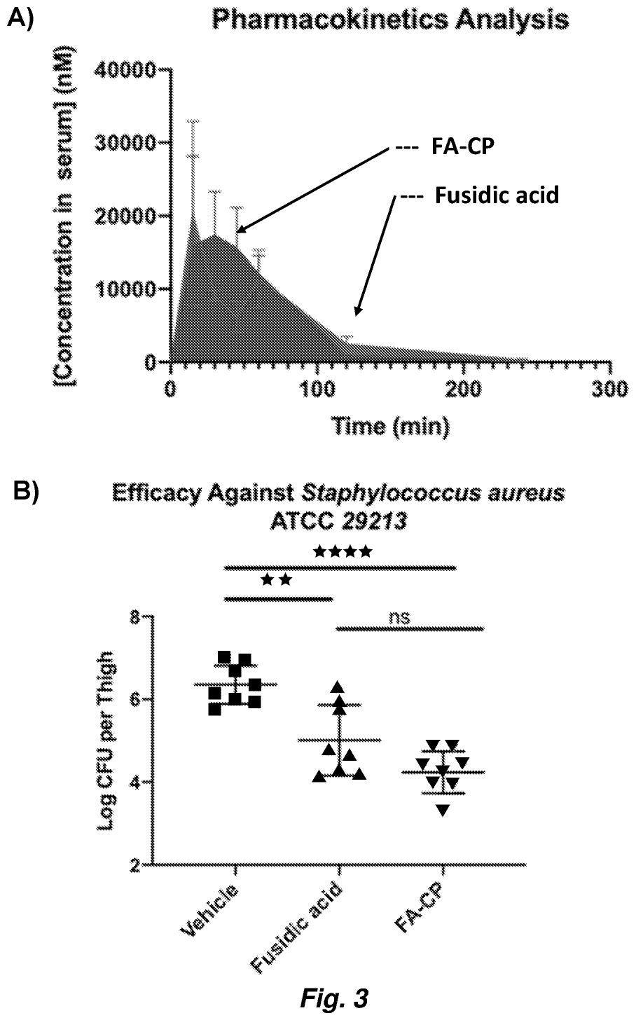

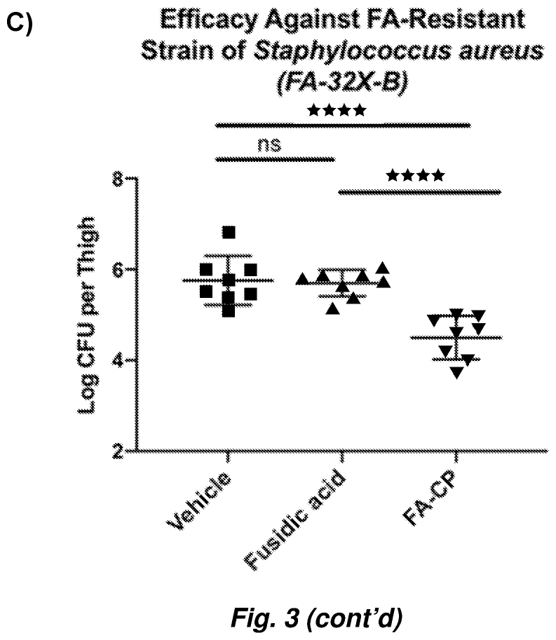

Pharmacokinetic studies and in vivo efficacy of FA and FA-CP. As a prelude to the mouse infection studies, the tolerability of FA (sodium fusidate) and FA-CP were assessed in mice at 50 mg/kg (intraperitoneal injection, once-a-day for 4 days), and both compounds were well tolerated by mice at this dose. Noteworthy is the rapid metabolism of FA in rodents, which is a major hurdle in the pre-clinical evaluation of FA analogues in mouse models. Head-to-head assessment of the pharmacokinetics of FA and FA-CP in mice revealed that these compounds have similar PK profiles (Table 9, FIG. 3A). The mouse and human PK profiles for FA are considerably different, as FA has a long half-life in humans of around 10-14 hours, with predominant metabolism by the liver; the rapid clearance observed in mice could be driven by the formation of 3-epiFA, a metabolite that has not been observed in humans.

TABLE 9
Pharmacokinetics of FA and FA-CP in mice.

| | | |
|---|---|---|
| Parameter | Estimate for Fusidic Acid | Estimate for FA-CP |

| | | | | |
|---|---|---|---|---|
| AUC | 561,683 | min*ng/ml | 701,474 | min*ng/ml |
| K10_HL | 31.2 | min | 19.0 | min |
| CL_F | 89.0 | ml/min/kg | 71.3 | ml/min/kg |
| Tmax | 5.4 | min | 26.4 | min |
| Cmax | 11,056.7 | ng/ml | 9,757.5 | ng/ml |

Encouraged by the efficacy of FA-CP against the panel of clinical isolates, the lack of toxicity to mammalian cells, and the promising tolerability and pharmacokinetic data, FA-CP was compared head-to-head to FA in in vivo efficacy studies; the design of these efficacy studies was guided by mouse models previously conducted with FA. First, a sensitive strain of S. aureus was assessed in a neutropenic thigh infection burden model utilizing S. aureus ATCC 29213, in which FA-CP and FA display the same MIC of 0.125 μg/mL. After infection, three doses of FA-CP and FA were administered (50 mg/kg, Intraperitoneal injection) 1 h, 2 h, 3 h post infection to separate groups of mice. Mice were sacrificed 24 hours post-infection and bacterial burden in the thigh muscle tissue homogenates was determined by serial dilution plating onto tryptic soy agar. Significant reductions in bacterial burden were observed with FA and FA-CP (FIG. 3B).

The improved potency of FA-CP against FA-resistant strains motivated and guided the second neutropenic thigh infection burden model. A FA-resistant S. aureus strain, FA-32×-B, generated at 32× the MIC of FA, was used for this study. FA has an MIC of 32 g/mL while FA-CP displays an MIC of 4 μg/mL (Table 5). After infection, three doses of FA-CP and FA were administered (50 mg/kg, intraperitoneal injection) 1 h, 2 h, 3 h post-infection to separate groups of mice. Mice were sacrificed 8 hours post-infection, and bacterial burden in the thigh muscle tissue homogenates was determined by serial dilution plating onto tryptic soy agar. This resulted in reductions in bacterial burden with FA-CP and no efficacy with FA (FIG. 3C). This study is, to our knowledge, the first mouse bacterial infection model in which a novel derivative of FA displays improved efficacy relative to FA.

### Discussion

Despite FA being approved in Europe since the 1960s to treat problematic S. aureus infections, there have been no follow-on drugs and FA remains the only member of the fusidane class to be used clinically. The high resistance frequency, drastic shifts in MIC upon bacterial resistance to FA, and lack of effective derivatives have narrowed the development path for this antibiotic class.

A considerable challenge in the creation of new and effective derivatives of FA has been a clouded structure-activity relationship for this compound, due to heterogeneity in the reported biological data for derivatives, with many compounds evaluated against the malaria parasite Plasmodium falciparum and Mycobacterium species but not against Gram-positive bacteria. In this work, we have constructed and assessed both previously synthesized derivatives and novel derivatives, allowing for a clearer SAR to emerge. Most notably, while previous investigations suggested that changes in the hydrophobic sidechain of FA diminished antibacterial activity, here we show that certain changes at this position are not only tolerable but lead to derivatives with favorable resistance profiles compared to FA.

Specifically, the reduced shift (relative to that observed for FA) in MIC upon resistance of S. aureus to FA-CP is an important feature of this compound. The highest MIC for the resistance mutants generated to FA-CP was 64 μg/mL, versus 256 μg/mL for resistance mutants generated to FA. In principle, this improvement could allow for administration of less FA-CP or similar compound. Although FA is considered a safe antibiotic, gastrointestinal side effects have been observed in 30-58% of patients treated with FA, and up to 30% of patients have elevated bilirubin.

Another interesting and potentially useful aspect of FA-CP is the ability of this compound to retain some activity against strains of S. aureus that are resistant to FA, including in a mouse model of infection. Specifically, FA-CP displayed in vivo efficacy against a FA-resistant S. aureus strain generated at 32× the MIC of FA (strain FA-32×-B). Our sequencing studies revealed that FA-32×-B possessed the mutation P406L. Noteworthy is the fact that this mutation has not only been observed in bacterial cell culture studies but has also been identified in clinical isolates of S. aureus, highlighting the potential of FA-CP to retain efficacy in vivo against some clinical isolates of S. aureus that are resistant to FA.

Furthermore, sequencing studies revealed some interesting differences between the mutational profile of bacteria arising from FA and FA-CP resistance. In our in-house studies, nine different amino acid changes were observed in EF-G for FA-resistant S. aureus, while only three different amino acid mutations in EF-G were observed for FA-CP-resistant S. aureus. FA mutations in EF-G have been classified into four different groups based on their role in resistance to FA: Group A (mutations affecting FA binding), Group B (mutations affecting EF-G ribosome interactions), Group C (mutations affecting EF-G conformation), and Group D (mutations affecting EF-G stability). Moreover, Group A mutations involve amino acids that are in direct contact with FA and with residues that can shape the drug pocket. Additionally, it has been postulated that Group C mutations are related to the interdomain orientations of EF-G, which can affect the FA-binding pocket along with the conformation dynamics and FA-locking of EF-G. The mutations for FA found in our studies have been previously classified and belong to Group A and Group C, whereas all the mutations observed for FA-CP have also been classified and belong to Group A, indicating that these mutations are in regions that can affect FA-binding.

While mutation to fusA, the gene encoding for EF-G, is a major mechanism of resistance for FA, it is important to note that other resistance mechanisms are operational in clinical isolates, specifically, the horizontal acquisition of plasmids harboring fusB or fusC. These resistance genes encode protective proteins that drive the dissociation of the EF-G ribosome complex from FA. Analogous to the shifts in MIC observed for FA upon mutations to the fusA gene, an increase in MIC has also been reported in clinical isolates that possess the fusB and/or fusC genes. For example, MIC ranges of 2-64 μg/mL in staphylococcal clinical isolates have been reported for clinical isolates that carry the fusC gene and MIC ranges of 4-1024 μg/mL have been reported for strains that possess the fusB gene. Due to the clinical relevance of strains that have acquired the fusC gene, the MIC of FA-CP was assessed against a strain that possesses this gene. FA-CP displays the same MIC as FA against S. aureus ATCC BAA-1721, with an MIC of 8 μg/mL. While resistance driven by the fusB and fusC genes is worrisome, the primary mechanism of clinical resistance to FA is still resistance mediated by mutations to the fusA gene. Specifically, mutation L461K found in clinical isolates of S. aureus is of high concern due its drastic shift in MIC (MIC>256 g/mL) upon a single amino acid mutation. Perhaps even more problematic are clinical isolates of S. aureus that are resistant to FA and possess four different EF-G amino acid alterations within the same strain. Five such clinical isolates have been isolated, and they all showed the following alterations in EF-G: V90I, H457Q, L461K, and A655V. All of these strains have an MIC greater than 256 μg/mL, highlighting the prominent role that mutations to the fusA gene play in the development of resistance to FA in the clinic.

FA-CP is the first analogue to outperform FA in a mouse infection model, which more broadly suggests the potential for certain FA derivatives. The important improvement in the resistance profile observed for FA-CP has some precedence in antibacterial drug discovery, for example in the dihydrofolate reductase inhibitor iclaprim and leucyl-tRNA synthetase inhibitor DS86760016, with improvement in each case likely due to subtly different modes of target engagement relative to the parent antibiotics (trimethoprim and GSK2251052, respectively). With access to derivatives now facile and the ability to improve upon its resistance profile now demonstrated, novel antibiotics of the fusidane class can be developed for the treatment of a variety of bacterial infections.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-bacterial agents and have higher potency and/or reduced toxicity as compared to fusidic acid. Preferably, compounds of the invention are more potent and less toxic than fusidic acid, and/or avoid a potential site of catabolic metabolism encountered with fusidic acid, i.e., have a different metabolic profile than fusidic acid.

The invention provides therapeutic methods of treating bacterial infections in a mammal, which involve administering to a mammal having a bacterial infection an effective amount of a compound or composition described herein. A mammal includes a primate, human, rodent, canine, feline, bovine, ovine, equine, swine, caprine, bovine and the like.

The ability of a compound of the invention to treat bacterial infections may be determined by using assays well known to the art. For example, the design of treatment protocols, toxicity evaluation, data analysis, quantification of bacterial cell kill are known. In addition, ability of a compound to treat bacterial infections may be determined using the Tests as described below.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Experimental Information for Biological Data and In Vivo Studies

Bacterial Strains. S. aureus ATCC 29213, E. faecium ATCC 19434, and S. aureus ATCC BAA-1721 were obtained from the American Type Culture Collection (ATCC). Resistant strains of E. faecium were obtained from the Centers for Disease Control and Prevention (CDC). Resistant strains of S. aureus were obtained from the Network on Antimicrobial Resistance in Staphylococcus aureus (NARSA) and the Centers for Disease Control and Prevention (CDC).

Antimicrobial Susceptibility Tests. Susceptibility testing was performed in biological triplicate, using the micro-dilution broth method as outlined by the Clinical and Laboratory Standards Institute. Bacteria were cultured with cation-adjusted Muller-Hinton broth (Sigma-Aldrich, Cat #90922) in round-bottom 96-well plates (Corning, Cat #3788). Human serum (Ultrafiltrate, unspecified gender, 30K Dalton membrane filtered) was purchased from BioIVT (Hicksville, NY).

Cell Culture. HFF-1 cells were obtained from ATCC. HFF-1 cells were grown in DMEM with 15% fetal bovine serum (Gemini Benchmark, Cat #100-106), 100 U/mL penicillin, and 100 μg/mL streptomycin. All cells were cultured at 37° C. in a 5% CO₂ environment. Media was prepared by the University of Illinois School of Chemical Sciences Cell Media Facility.

Cell Viability. Cells were harvested, seeded in a 96 well-plate and allowed to adhere. Cells were treated with investigational compounds in DMSO (1% final concentration). Cells were incubated for 72 h before viability was assessed by the Alamar Blue Assay. Raptinal (20 M) was used as a dead control.

Mouse Liver Microsome Stability Assay. A mixture of PBS (pH 7.4), NADPH regenerating system solution A (Corning Life Sciences), and NADPH regenerating system solution B (Corning Life Sciences) was incubated at 37° C. in a shaking incubator for 5 min. Next, compound was added in DMSO (final concentration 50 μM, 0.5% DMSO) before ice-cold mouse liver microsomes (Thermo Fisher, male CD-1 mice, pooled) were added (final protein concentration of 1 mg/mL). An aliquot was immediately removed, quenched with an equal volume of 100 μM internal standard and centrifuged at 13,000 rcf for 3 min. The reactions were incubated at 37° C. in a shaking incubator for 3 h. A second aliquot was removed and quenched. Samples were analyzed with the 5500 QTRAP LC/MS/MS system (Sciex, Framingham, MA) in Metabolomics Lab of Roy J. Carver Biotechnology Center, University of Illinois at Urbana-Champaign. Software Analyst 1.6.2 was used for data acquisition and analysis. The 1200 series HPLC system (Agilent Technologies, Santa Clara, CA) includes a degasser, an autosampler, and a binary pump. The LC separation was performed on an Agilent Sb-Aq column (4.6×50 mm, 5 m) with mobile phase A (0.1% formic acid in water) and mobile phase B (0.1% formic acid in acetonitrile). The flow rate was 0.3 mL/min. The linear gradient was as follows: 0-3 min, 100% A; 10-16 min, 5% A; 16.5-22 min, 100% A. The autosampler was set at 10° C. The injection volume was 1 μL. Mass spectra were acquired under both positive (ion spray voltage was +5500 V) and negative (ion spray voltage was −4500 V) electrospray ionization (ESI). The source temperature was 450° C. The curtain gas, ion source gas 1, and ion source gas 2 were 33, 65, and 60 psi, respectively. Multiple reaction monitoring (MRM) was used for quantitation.

Selection of Resistant Mutants. Resistant mutants were selected via the large inoculum method. Briefly, S. aureus ATCC 29213 (1×10⁹ CFU) were plated on 100 mm plates of LB agar containing 4, 2, 1, 0.5, and 0.25 μg/mL. Colonies were visible after incubation at 37° C. for 24 h. Resistant colonies were confirmed by streaking on selective media with the same concentration of fusidic acid and compounds 16, FA-CP (25), 26, 27.

Sequencing of fusA. FusA was amplified by colony PCR. Colonies were picked and diluted in 100 μL sterile H₂O. PCR reactions are setup by combining MiFi Mix (Bioline, London, UK), 20 μM primer mix [fusA-F2, fusA_seq1, Forward EF-G2, and Reverse EF-G](S. aureus ATCC 29213), template DNA, and H₂O. Reactions were performed on C1000 Thermal Cycler (Bio-Rad, Hercules, CA) with the following conditions: 5 minutes denaturation at 95° C., followed by 30 cycles of 20 seconds at 95° C., 20 seconds at 50° C., and either 1 minute (fusA1) or 1.5 minutes (fusA2) at 72° C. A 10 μL portion of the PCR reaction mixture was analyzed by agarose gel to confirm the product. PCR reactions were purified using GeneJET PCR Purification Kit (Thermo Scientific). PCR amplicons were submitted to the Core DNA Sequencing Facility at the University of Illinois at Urbana-Champaign for Sanger sequencing with the following primers to sequence the fusA [fusA2, fusA_seq1, Forward EF-G2, Forward EF-G3, Forward EF-G4] (Table 10).

TABLE 10
Primers for sequencing of fusA gene in S. aureus (Int. J. Med. Microbiol.
2012, 302 (2), 96).

| Portion | Elongation | | |
|---|---|---|---|
| of fusA | Factor G | | |
| Gene | Domain (D) | Oligonucleotide | Sequence |
| fusA1 | D1 | fusA-F2 | 5′-CTC GTA ATA TCG GTA |
| | | | TCA TG-3′ (SEQ ID NO: 1) |
| fusA1 | D1 | fusA_seq1 | 5′-TAA GGG TCA GTC ATA |
| | | | ACT TT-3′ (SEQ ID NO: 2) |
| fusA1 | D1 | Forward EF-G2 | 5′-TGA TCG TTT ACA AGC |
| | | | TAA CGC-3′ (SEQ ID NO: 3) |
| fusA2 | D1 and D5 | Forward EF-G2 | 5′-TGA TCG TTT ACA AGC |
| | | | TAA CGC-3′ (SEQ ID NO: 3) |
| fusA2 | D1 and D5 | Reverse EF-G | 5′-AGA AAT TAT TTA TAG |
| | | | CGA TGC-3′ (SEQ ID NO: 4) |
| fusA2 | D1 and D5 | Forward EF-G3 | 5′-ATT CTT CCG TGT GTA |
| | | | CTC AGG-3′ (SEQ ID NO: 5) |
| fusA2 | D1 and D5 | Forward EF-G4 | 5′-TGG TCA ATA CGG TGA |
| | | | TGT TCA-3′ (SEQ ID NO: 6) |

Mouse MTD of Sodium fusidate and FA-CP (25). The protocol was approved by the IACUC at the University of Illinois at Urbana-Champaign (Protocol Number: 16144 and 19181). In these studies, 10- to 12-week-old female C57BL/6 mice purchased from Charles River were used. The maximum tolerated dose (MTD) of single compound was determined first. Sodium fusidate and FA-CP (25) were formulated in 5% DMSO, 10% Tween 20, 85% PBS. Sodium fusidate and FA-CP (25) were given by IP injection. All the mice were monitored for signs of toxicity for 2 weeks. For multiple doses, the compound was given by daily IP for 4 consecutive days, and mice were monitored for signs of toxicity for 1 month. MTD was the highest dosage with acceptable toxicity (e.g., <20% weight loss). Sodium fusidate and FA-CP (25) were well tolerated as a single dose of 50 mg/kg. Further analysis showed that sodium fusidate and FA-CP (25) were well tolerated with daily dosing of 50 mg/kg for 4 consecutive days. The MTD of FA-CP (25) was used to inform the dosing schedule used in subsequent efficacy studies.

Pharmacokinetic Assessment of sodium fusidate and FA-CP (25). The protocol was approved by the IACUC at the University of Illinois at Urbana-Champaign (Protocol Number: 16144, 19181). In these studies, 10- to 12-week-old female C57BL/6 mice purchased from Charles River were used. The compounds were formulated in 5% DMSO, 10% Tween 20, and 85% PBS. Mice were treated with sodium fusidate or FA-CP (25) (50 mg/kg) via intraperitoneal injection with three mice per time point (15, 30, 45, 60, 120, and 240 min). At specific time points, mice were sacrificed, blood was collected and centrifuged, and the serum was frozen at −80° C. until analysis. The proteins in a 10 μL aliquot of serum were precipitated by the addition of 50 μL acetonitrile with the addition of 10 μL of 1.6 ug/mL internal standard (sodium fusidate was the internal standard when measuring FA-CP (25) and FA-CP (25) was the internal standard when measuring sodium fusidate). The sample was then vortexed and centrifuged to remove the proteins. Supernatants were analyzed with the QTRAP 5500 LC/MS/MS system (Sciex) in the Metabolomics Laboratory of the Roy J. Carver Biotechnology center, University of Illinois at Urbana-Champaign. Software Analyst 1.6.2 was used for data acquisition and analysis. The 1200 Series HPLC System (Agilent Technologies) includes a degasser, an autosampler and a binary pump. The liquid chromatography separation was performed on an Agilent Zorbax SB-Aq column (4.6 mm×50 mm; 5 μm) with mobile phase A (0.1% formic acid in water) and mobile phase B (0.1% formic acid in acetonitrile). The flow rate was 0.3 mL/min. The linear gradient was as follows: 0-1 min: 95% A; 8-13 min: 0% A; 8.1-18.5 min: 95% A. The autosampler was set at 10° C. The injection volume was 5 μL. Mass spectra were acquired under negative electrospray ionization with a voltage of −4,500 V. The source temperature was 450° C. The curtain gas, ion source gas 1 and ion source gas 2 were 32, 60 and 60 psi, respectively. Multiple reaction monitoring was used for quantitation: fusidic acid: m/z 515.3-->m/z 393.3, FA-CP (25) m/z 541.4-->m/z 437.2. The limit of quantitation of (S/N=10) was 1 nM. Pharmacokinetic parameters were calculated with a one-compartment model using a nonlinear regression program (Phoenix WinNonlin Version 8.1; Certara USA).

Neutropenic Thigh Infection Burden Study with Sodium fusidate and FA-CP (25). Mouse studies were carried out in strict accordance with the recommendations in the guide for the Care and Use of Laboratory Animals of the National Institutes of Health. Animal protocol was approved by the Institutional Animal Care and Use Committee (IACUC) at the University of Illinois at Urbana-Champaign (protocol #17271). Briefly, seven-week-old male CD1 mice (cohorts of 8) were rendered neutropenic by intraperitoneal injection of cyclophosphamide (150 mg/kg on Day-4 to Day-2 and 100 mg/kg Day-1). On Day-1, mice were anesthetized with a combination of xylazine/ketamine, and furs on the right hind thigh of were removed by clipping with a pair of scissors followed by application of depilating gel (Veet Aloe Vera Legs & Body Hair Remover Gel Cream). After 24 hours, mice were anesthetized with isoflurane, and infected with S. aureus strain ATTC 29213 or the S. aureus FA-resistant strain 32×-B at concentration of ˜1×10\hat{ }6 CFUs (in 50 μl) by injection into the thigh muscle (bicep femoris) with a 25G ⅚″ needle. Infected mice were intraperitoneally-treated with vehicle (85% PBS, 10% Tween, 5% DMSO), 50 mg/kg of sodium fusidate and FA-CP (25), at 1, 2, 3 hours post-infection (hpi) individually in 100 μl volume. Infected animals were monitored for myositis and lameness until euthanasia. At indicated times (24-hpi for the 50 mg/kg cohorts for the S. aureus 29213 infection model, and 8-hpi for the 50 mg/kg cohorts for the S. aureus (FA-32×-B) infection model, mice were euthanized with CO₂ asphyxiation from a compressed gas source followed by cervical dislocation. Infected thigh muscle tissues were harvested and homogenized with a Omni Soft Tissue Tip™ Homogenizer (OMNI International) in 2 ml of sterile PBS. Bacterial burden in the tissue homogenates were determined by serial dilution plating onto tryptic soy agar.

### Example 2. Materials and Methods for Synthesis of Derivatives

Fusidic acid was purchased from J&K Scientific, Ltd. and Hebei Shengmei Medical Technology Co. Ltd. Sodium fusidate was purchased from Alfa Aesar and AvaChem Scientific. Other chemical reagents were purchased from commercial sources and used without further purification. Anhydrous solvents used during these studies were dried after being passed through columns with activated alumina under nitrogen using a PureSolv MD-5 (Inert previously Innovative Technology, Inc.) solvent purification system. Flash chromatography was performed using silica gel (230-400 mesh).

Various NMR experiments were conducted in the NMR facilities at UIUC. ¹H NMR and ¹³C NMR experiments were recorded on Varian Unity spectrometers at 500 MHz and 125 Hz, respectively and/or a Bruker Avance III HD 500 MHz NMR system equipped with a CryoProbe. Spectra were obtained in the following solvents (reference peaks also included for ¹H and ¹³C NMRs): CDCl₃ (¹H NMR: 7.26 ppm; ¹³C NMR: 77.20 ppm), and CD₃OD (¹H NMR: 3.31 ppm; ¹³C NMR: 49.10 ppm). NMR experiments were performed at room temperature unless otherwise indicated. Chemical shift values are reported in parts per million (ppm) for all ¹H NMR and ¹³C NMR spectra. ¹H NMR multiplicities are reported as: s=singlet, d=doublet, t=triplet, q=quartet, m=multiplet, br=broad, pentet=pent., sextet, =sex, heptet=hept. High-resolution mass spectra were obtained using Waters Q-TOF Ultima ESI and Agilent 6230 ESI TOF LC/MS spectrometers.

Final compounds for biological assays were purified using a Teledyne ISCO ACCQPrep HP125 Preparative HPLC (Column: Teledyne ISCO RediSep Prep C18-20 mm×250 mm, 100 Å, 5-micron).

Purity of final compounds assessed for biological activity were purified to >95% as assessed by an Agilent 6230 LC/MS TOF (Column: Agilent ZORBAX Eclipse Plus C18 Rapid Resolution HT 2.1×100 mm, 1.8-micron) and/or and Agilent Technologies 1290 Infinity II UHPLC equipped with a Phenomenex Kinetex column (2.1 mm ID×50 mm, 1.7-micron particle size, 100 Å pore size).

Preparation and Characterization of Core Fusidic Acid Analogues.

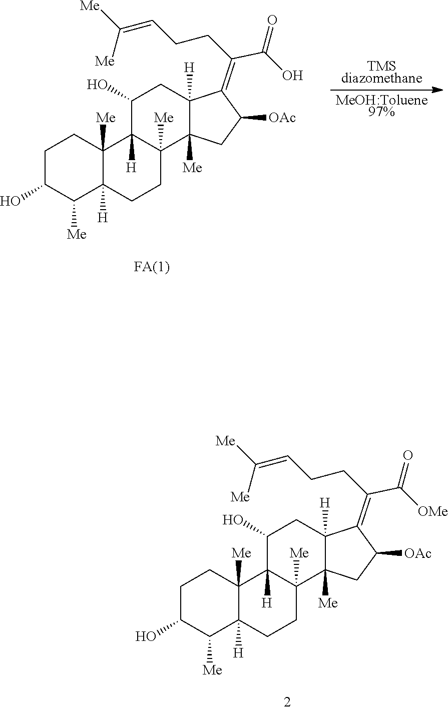

Experimental procedure and spectra for compound 2 have been previously reported (Nat. Chem. 2017, 9 (12), 1213).

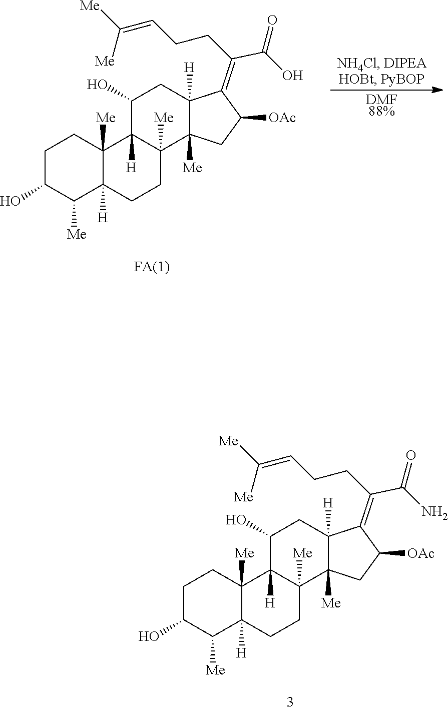

Experimental procedure and spectra for compound 3 have been previously reported (Medchemcomm 2015, 6 (11), 2023).

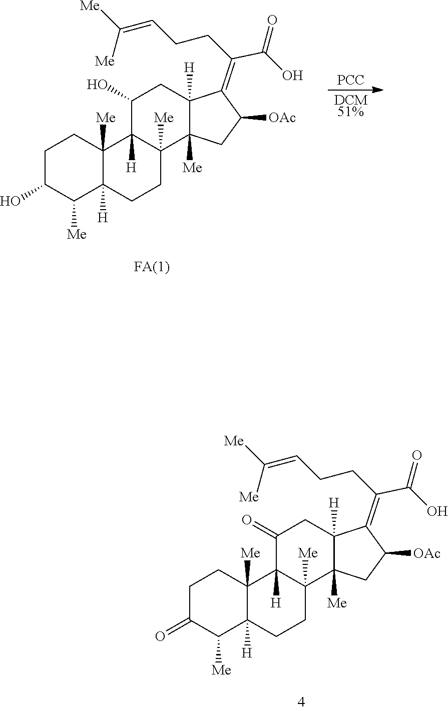

Procedure for (Z)-2-((4S,5S,8S,9S,10S,13R,14S,16S)-16-hydroxy-4,8,10,14-tetramethyl-3,11-dioxohexadecahydro-17H-cyclopenta[a]phenanthren-17-ylidene)-6-methylhept-5-enoic acid (4). In a 500 mL RB flask, FA (3.00 g, 5.80 mmol, 1.0 eq.) was dissolved in DCM (200 mL). PCC (3.12 g, 14.5 mmol, 2.5 eq.) was added to the reaction. The reaction was then stirred at room temperature for 2 hrs. The solution was then concentrated, and the reaction was filtered through celite using EtOAc as the solvent. The eluent was then collected and concentrated. The resulting crude product was then purified by silica gel column chromatography (100% EtOAc) followed by preparatory HPLC (C18 20×250 mm, 10-100% ACN in H₂O) to afford 4 (1.53 g, 51%) as a white solid.

¹H NMR (500 MHz, Chloroform-d) δ 5.92 (d, J=8.3 Hz, 1H), 5.10-5.05 (m, 1H), 2.96-2.86 (m, 2H), 2.77-2.65 (m, 2H), 2.60 (s, 1H), 2.54-2.47 (m, 1H), 2.44-2.31 (m, 3H), 2.26-2.08 (m, 3H), 2.08-1.99 (m, 3H), 1.98 (s, 3H), 1.84-1.77 (m, 1H), 1.66 (s, 3H), 1.58 (s, 3H), 1.58-1.55 (m, 1H), 1.45 (d, J=14.3 Hz, 1H), 1.25-1.20 (m, 1H), 1.19 (s, 3H), 1.18-1.16 (m, 1H), 1.15 (s, 3H), 1.07-1.03 (m, 6H). (43 non-exchangeable protons).

¹³C NMR (125 MHz, Chloroform-d) δ 216.02, 209.69, 174.27, 170.44, 148.46, 133.38, 130.96, 122.60, 74.32, 58.42, 48.83, 47.21, 46.36, 44.95, 44.53, 40.90, 38.22, 36.85, 36.74, 33.45, 32.55, 28.93, 28.16, 25.86, 24.11, 22.65, 21.35, 20.69, 17.91, 17.19, 14.25.

HRMS(ESI): m/z calc. for C₃₁H₄₄O₆ [M]+: 513.3216. found: 513.3228.

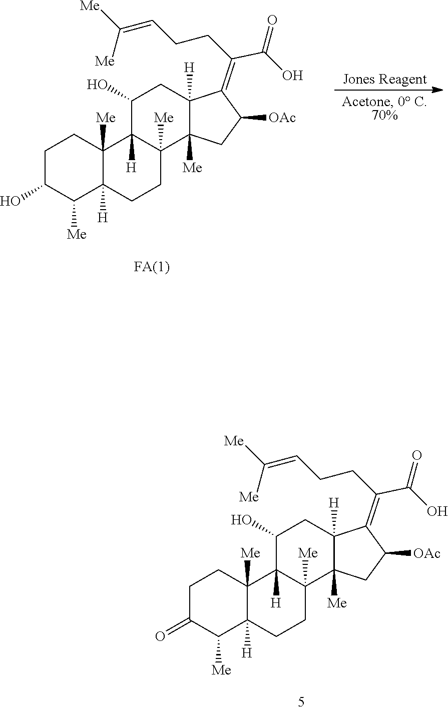

Experimental procedure and spectra for compound 5 have been previously reported (ACS Infect. Dis. 2019, 5 (9), 1634).

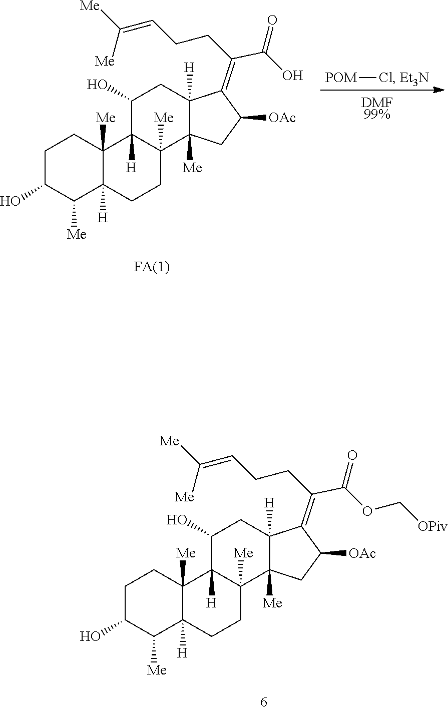

Experimental procedure and spectra for compound 6 have been previously reported (J. Chem. Inf. Model 2018, 58 (8), 1553; J. Med. Chem. 2006, 49 (5), 1503).

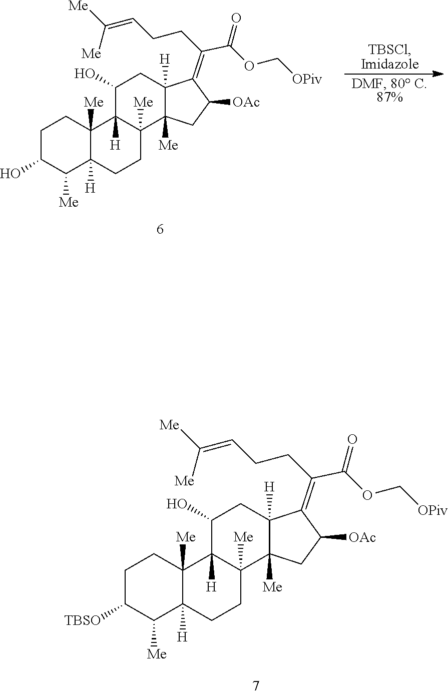

Procedure for (pivaloyloxy)methyl(Z)-2-((3R,4S,5S,8S,9S,10S,11R,13R,14S,16S)-16-acetoxy-3-((tert-butyldimethylsilyl)oxy)-11-hydroxy-4,8,10,14-tetramethylhexadecahydro-17H cyclopenta[a]phenanthren-17-ylidene)-6-methylhept-5-enoate (7). In a 25 mL round bottom flask, 6 (2.08 g, 3.29 mmol, 1.0 eq.) was dissolved in DMF (10 mL). Then imidazole (526 mg, 7.72 mmol, 2.3 eq.) and TBSCl (1.01 g, 7.72 mmol, 2.3 eq.) were added, respectively. The reaction was then heated to 80° C. for 6 hrs. EtOAc and brine were then added, and the layers were separated. The aqueous layer was extracted three times with EtOAc. The combined organic layers were dried over MgSO₄, filtered, and concentrated. Silica gel column chromatography (5:1 Hex/EtOAc) afforded 7 as a white solid (2.14 g, 87%).

¹H NMR (500 MHz, Chloroform-d) δ 5.85 (d, J=8.3 Hz, 1H), 5.78 (d, J=5.4 Hz, 1H), 5.69 (d, J=5.4 Hz, 1H), 5.09-5.04 (m, 1H), 4.34-4.28 (m, 1H), 3.70-3.66 (m, 1H), 3.07-3.00 (m, 1H), 2.50-2.36 (m, 2H), 2.34-2.28 (m, 1H), 2.24-2.07 (m, 4H), 1.96 (s, 3H), 1.83-1.71 (m, 3H), 1.66 (s, 3H), 1.58 (s, 3H), 1.54-1.46 (m, 3H), 1.45-1.38 (m, 2H), 1.34 (s, 3H), 1.29 (d, J=14.2 Hz, 1H), 1.24 (s, 1H), 1.19 (s, 9H), 1.12-1.01 (m, 2H), 0.94 (s, 3H), 0.89 (s, 12H), 0.80 (d, J=6.7 Hz, 3H), 0.01 (d, J=6.4 Hz, 6H). (71 non-exchangeable protons).

¹³C NMR (125 MHz, Chloroform-d) δ 177.18, 170.39, 168.25, 151.33, 132.77, 129.42, 123.10, 79.90, 74.45, 72.06, 68.52, 49.19, 49.00, 44.53, 39.61, 39.15, 38.90, 37.34, 36.67, 36.40, 35.43, 33.44, 30.99, 30.72, 28.92, 28.38, 27.04(3C), 26.06(3C), 25.87, 24.46, 22.63, 20.98, 20.53, 18.30, 18.27, 17.94, 16.92, −4.19, −4.72.

HRMS(ESI): m/z calc. for C₄₃H₇₂O₈NaSi [M+Na]+: 767.4894. found: 767.4921

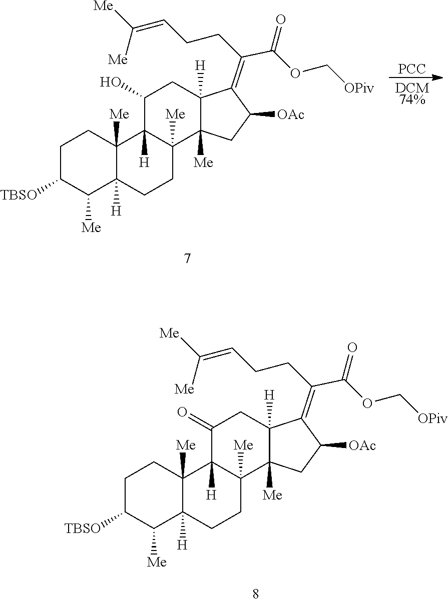

Procedure for (pivaloyloxy)methyl (Z)-2-((3R,4S,5S,8S,9S,10S,13R,14S,16S)-16-acetoxy-3-((tert-butyldimethylsilyl)oxy)-4,8,10,14-tetramethyl-11-oxohexadecahydro-17H-cyclopenta[a]phenanthren-17-ylidene)-6-methylhept-5-enoate (8). In a 65 mL RB flask, 7 (1.71 g, 2.37 mmol, 1.0 eq.) was dissolved in DCM (34.0 mL). PCC (1.48 g, 6.87 mmol, 3.0 eq.) was added to the reaction. The reaction was then stirred at room temperature for 2 hrs. The reaction was the filtered and washed with EtOAc. The eluent was concentrated. The resulting crude product was then purified by silica gel column chromatography (3:1 Hex/EtOAc) afforded 8 as a white solid (1.26 g, 74%).

¹H NMR (500 MHz, Chloroform-d) δ 5.87 (d, J=8.3 Hz, 1H), 5.77 (d, J=5.4 Hz, 1H), 5.70 (d, J=5.4 Hz, 1H), 5.05-5.01 (m, 1H), 3.70-3.67 (m, 1H), 2.88 (dd, J=13.1, 4.8 Hz, 1H), 2.84-2.79 (m, 1H), 2.67-2.60 (m, 1H), 2.50 (s, 1H), 2.40-2.28 (m, 2H), 2.23-2.18 (m, 1H), 2.17-2.08 (m, 2H), 2.07-2.00 (m, 1H), 1.98 (s, 3H), 1.97-1.83 (m, 2H), 1.76-1.69 (m, 1H), 1.64 (s, 3H), 1.60-1.58 (m, 1H), 1.56 (s, 3H), 1.55-1.52 (m, 1H), 1.49-1.42 (m, 1H), 1.39 (d, 1H), 1.24 (s, 1H), 1.19 (s, 9H), 1.15 (s, 3H), 1.13 (s, 3H), 0.98 (s, 3H), 0.97-0.90 (m, 1H), 0.88 (s, 9H), 0.86-0.84 (m, 1H), 0.78 (d, J=6.7 Hz, 3H), 0.00 (d, J=9.6 Hz, 6H).

¹³C NMR (125 MHz, Chloroform-d) δ 210.33, 177.12, 170.15, 167.72, 148.48, 133.19, 130.41, 122.63, 79.89, 74.29, 72.35, 58.84, 48.94, 47.40, 44.81, 41.00, 38.89, 38.46, 38.33, 37.94, 35.26, 33.54, 31.02, 28.95, 28.17, 27.95, 27.01(3C), 26.03(3C), 25.82, 23.26, 21.07, 20.90, 20.21, 18.25, 17.90, 17.23, 17.03, −4.24, −4.74.

HRMS(ESI): m/z calc. for C₄₃H₇₀O₈NaSi [M+Na]+: 765.4738. found: 765.4743.

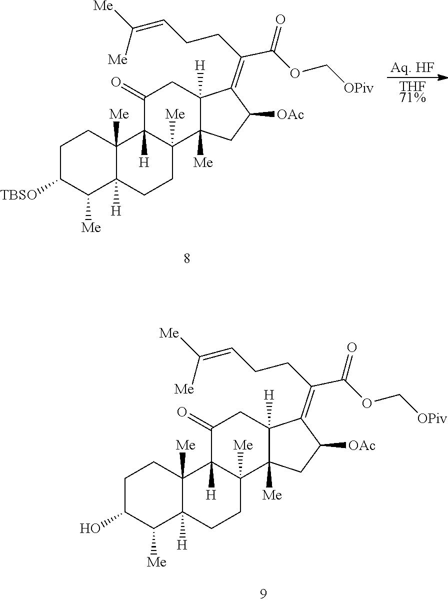

Procedure for (pivaloyloxy)methyl (Z)-2-((3R,4S,5S,8S,9S,10S,13R,14S,16S)-16-acetoxy-3-hydroxy-4,8,10,14-tetramethyl-11-oxohexadecahydro-17H-cyclopenta[a]phenanthren-17-ylidene)-6-methylhept-5-enoate (9). In a 25 mL polystyrene bottle, 8 (161.8 mg, 0.217 mmol, 1.0 eq.) was dissolved in THF (10.0 mL). Aqueous HF (48% by weight) (1.50 mL) was added to the reaction. The reaction was then stirred at room temperature for 14 hrs. An additional (1.50 mL) of aqueous HF were added, and the reaction was stirred for an additional 8 hrs. The reaction was then quench by the addition of 10% NaOH. The solution was then transfer to a polystyrene bottle where the reaction was extracted three times using DCM. The combined organic layers were dried over MgSO₄, filtered, and concentrated. Silica gel column chromatography (3:1 Hex/EtOAc) afforded 9 as a white solid (97.2 mg, 71%).

¹H NMR (500 MHz, Chloroform-d) δ 5.85 (d, J=8.3 Hz, 1H), 5.76 (d, J=5.4 Hz, 1H), 5.68 (d, J=5.4 Hz, 1H), 5.05-5.00 (m, 1H), 3.75-3.72 (m, 1H), 2.91-2.85 (m, 1H), 2.83-2.78 (m, 1H), 2.68-2.60 (m, 1H), 2.55 (s, 1H), 2.38-2.27 (m, 2H), 2.22-2.10 (m, 2H), 2.07-1.98 (m, 2H), 1.97 (s, 3H), 1.96-1.81 (m, 4H), 1.68-1.65 (m, 1H), 1.63 (s, 3H), 1.55 (s, 3H), 1.53-1.46 (m, 2H), 1.38 (d, 1H), 1.23-1.19 (m, 1H), 1.18 (s, 9H), 1.14 (s, 3H), 1.13 (s, 3H), 1.05-0.99 (m, 1H), 0.99 (s, 3H), 0.88 (d, J=6.9 Hz, 3H). (55 non-exchangeable protons).

¹³C NMR (125 MHz, Chloroform-d) δ 210.21, 177.09, 170.18, 167.69, 148.21, 133.18, 130.36, 122.61, 79.88, 74.26, 71.50, 58.68, 48.87, 47.66, 44.90, 41.19, 38.86, 38.48, 38.30, 37.85, 34.81, 32.73, 30.32, 28.89, 28.26, 27.92, 26.98(3C), 25.79, 23.24, 21.15, 20.86, 20.44, 17.87, 17.19, 16.16.

HRMS(ESI): m/z calc. for C₃₇H₅₆O₈Na [M+Na]+: 651.3873. found: 651.3878.

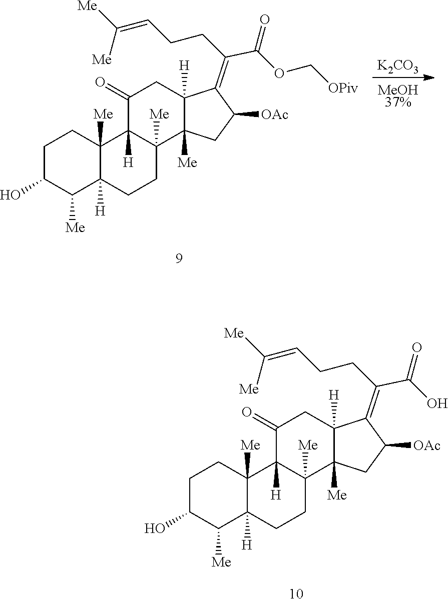

Procedure for (Z)-2-((3R,4S,5S,8S,9S,10S,13R,14S,16S)-16-acetoxy-3-hydroxy-4,8,10,14-tetramethyl-11-oxohexadecahydro-17H-cyclopenta[a]phenanthren-17-ylidene)-6-methylhept-5-enoic acid (10). In a 5 mL vial, 9 (97.2 mg, 0.15 mmol, 1.0 eq.) was dissolved in MeOH (1.22 mL). K₂CO₃ (42.7 mg, 0.30 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 10 (29.3 mg, 37%) as a white solid.

¹H NMR (500 MHz, Methanol-d₄) δ 5.80 (d, J=8.3 Hz, 1H), 5.19-5.14 (m, 1H), 3.72-3.68 (m, 1H), 2.91-2.85 (m, 1H), 2.76-2.70 (m, 3H), 2.36-2.26 (m, 2H), 2.20-2.03 (m, 5H), 2.03 (s, 3H), 2.02-1.97 (m, 2H), 1.91-1.83 (m, 1H), 1.67 (s, 3H), 1.66-1.63 (m, 1H), 1.62 (s, 3H), 1.57-1.50 (m, 1H), 1.32 (d, J=13.3 Hz, 1H), 1.26-1.22 (m, 1H), 1.22 (s, 3H), 1.21-1.19 (m, 1H), 1.18 (s, 3H), 1.11-1.03 (m, 1H), 1.03 (s, 3H), 0.89 (d, J=6.8 Hz, 3H). (44 non-exchangeable protons).

¹³C NMR (125 MHz, Methanol-d₄) δ 213.77, 178.91, 173.30, 140.61, 135.90, 132.70, 125.24, 75.88, 72.36, 59.98, 50.17, 47.66, 46.46, 42.75, 39.79, 39.49, 38.97, 36.32, 34.02, 31.25, 31.07, 29.75, 28.79, 25.98, 23.50, 21.66, 21.49, 21.17, 17.95, 17.42, 16.66.

HRMS(ESI): m/z calc. for C₃₁H₄₆O₆Na [M+Na]+: 537.3192. found: 537.3192.

### Example 3. Preparation and Characterization of Fusidic Acid Side Chain Analogues

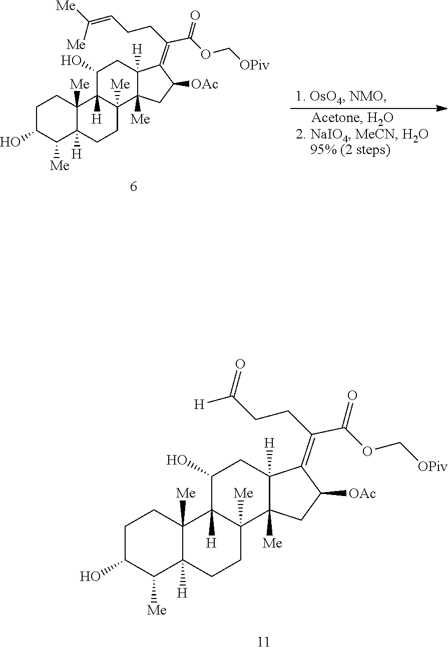

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-oxopentanoate (11). In a 65 mL round bottom flask, 6 (980 mg, 1.55 mmol, 1.0 eq.) was dissolved in acetone (9.25 mL) and distilled H₂O (0.80 mL). Then NMO (281 mg, 2.39 mmol, 1.5 eq.) and OsO₄ (0.15 mL, 0.028 mmol, 0.02 eq.) in a 0.2 M solution in MeCN were added, respectively. The solution was then stirred at room temperature under a N2 atmosphere for 1 hr. The reaction was then quench by the addition of 10 mL of saturated sodium thiosulfate. The reaction was stirred for an additional 45 minutes. The reaction was then concentrated in vacuo to remove the acetone. EtOAc and brine were then added, and the layers were separated. The aqueous layer was extracted five times with EtOAc. The combined organic layers were dried over MgSO₄, filtered, and concentrated. Silica gel column chromatography (100% EtOAc) afforded the crude dihydroxylation intermediate that was used without further purification.

In a 50 mL round bottom flask, the dihydroxylation intermediate (1.02 g, 1.53 mmol, 1.0 eq.) was dissolved in MeCN (10.0 mL) and distilled H₂O (10.0 mL). Then NaIO₄ (673 mg, 3.14 mmol, 2.0 eq.) was added. The solution was then stirred at room temperature for 6 hrs. The reaction was then cooled 0° C. and quenched by the addition of 10 mL of saturated sodium sulfite. The reaction was stirred for an additional 45 minutes. EtOAc and brine were then added, and the layers were separated. The aqueous layer was extracted three times with EtOAc. The combined organic layers were dried over MgSO₄, filtered, and concentrated. Silica gel column chromatography (100% EtOAc) afforded 11 as a white solid (895 mg, 95%).

¹H NMR (500 MHz, Chloroform-d) δ 9.73 (s, 1H), 5.85 (d, J=8.3 Hz, 1H), 5.77 (d, J=5.4 Hz, 1H), 5.69 (d, J=5.4 Hz, 1H), 4.35-4.31 (m, 1H), 3.74-3.70 (m, 1H), 3.10-3.04 (m, 1H), 2.75-2.65 (m, 3H), 2.55-2.45 (m, 1H), 2.31-2.23 (m, 1H), 2.19-2.08 (m, 4H), 1.96 (s, 3H), 1.85-1.78 (m, 2H), 1.74-1.66 (m, 2H), 1.56-1.51 (m, 2H), 1.51-1.43 (m, 1H), 1.35 (s, 3H), 1.29 (d, J=13.6 Hz, 1H), 1.20 (s, 9H), 1.15-1.05 (m, 2H), 0.96 (s, 3H), 0.91-0.88 (m, 6H). (50 non-exchangeable protons).

¹³C NMR (125 MHz, Chloroform-d) δ 201.23, 177.25, 170.29, 167.55, 153.29, 127.35, 80.06, 74.40, 71.58, 68.20, 49.49, 49.01, 44.74, 43.96, 39.66, 39.05, 38.93, 36.95, 36.75, 35.84, 35.69, 31.93, 30.14, 29.99, 27.03(3C), 23.90, 23.43, 21.47, 21.09, 20.94, 18.05, 16.14.

HRMS(ESI): m/z calc. for C₃₄H₅₂O₉Na [M+Na]+: 627.3509. found: 627.3497.

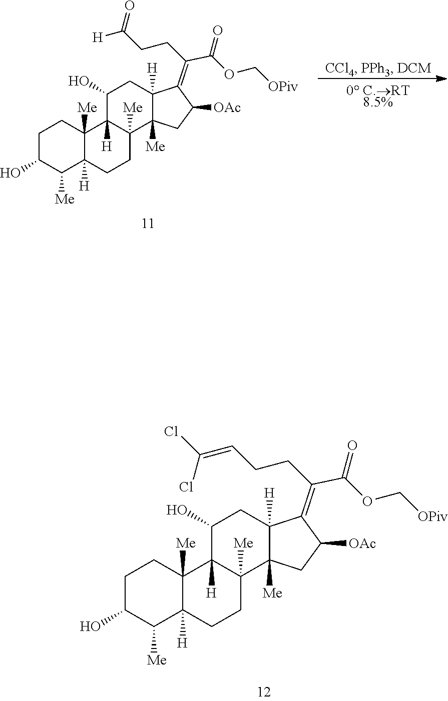

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-dichlorohex-5-enoate (12). In a 10 mL round bottom flask, CCl₄ (0.18 mL, 1.85 mmol, 2.0 eq.) was dissolved in DCM (3.00 mL). The solution was then cooled to 0° C. This was followed by the addition of PPh₃ (967 mg, 3.68 mmol, 4.0 eq.). The reaction was then allowed to stirred at 0° C. for 20 minutes. Compound 11 (556 mg, 0.92 mmol, 1.0 eq.) was then added and the reaction was stirred at 0° C. for 3 hrs. The reaction was then stirred at room temperature for 12 hrs. EtOAc and brine were then added, and the layers were separated. The aqueous layer was extracted three times with EtOAc. The combined organic layers were dried over MgSO₄, filtered, and concentrated. Silica gel column chromatography (1:1 Hex/EtOAc) afforded 12 as a white solid (52.6 mg, 8.5%).

¹H NMR (500 MHz, Chloroform-d) δ 5.87 (d, J=8.3 Hz, 1H), 5.84 (t, J=7.4 Hz, 1H), 5.79 (d, J=5.4 Hz, 1H), 5.69 (d, J=5.4 Hz, 1H), 4.36-4.33 (m, 1H), 3.76-3.72 (m, 1H), 3.11-3.05 (m, 1H), 2.55-2.51 (m, 2H), 2.36-2.08 (m, 7H), 1.97 (s, 3H), 1.89-1.80 (m, 2H), 1.77-1.71 (m, 2H), 1.63-1.57 (m, 3H), 1.53-1.47 (m, 1H), 1.37 (s, 3H), 1.30 (d, J=13.4 Hz, 1H), 1.21 (s, 9H), 1.14-1.09 (m, 1H), 0.97 (s, 3H), 0.92-0.90 (m, 6H). (50 non-exchangeable protons).

¹³C NMR (125 MHz, Chloroform-d) δ 177.21, 170.34, 167.70, 153.25, 128.50, 127.83, 121.15, 80.05, 74.41, 71.54, 68.28, 49.37, 49.03, 44.74, 39.64, 39.09, 38.94, 37.18, 36.43, 36.21, 35.88, 32.45, 30.38, 30.10, 29.82, 27.24, 27.06(3C), 24.28, 23.03, 20.97, 20.93, 18.10, 16.12.

HRMS(ESI): m/z calc. for C₃₅H₅₂O₈NaCl₂ [M+Na]+: 693.2937. found: 693.2946.

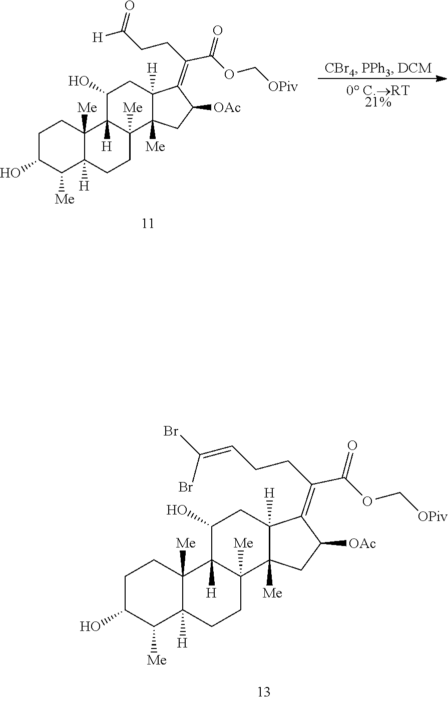

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-dibromohex-5-enoate (13). In a 65 mL round bottom flask, CBr₄ (1.56 g, 4.70 mmol, 3.0 eq.) was dissolved in DCM (38.0 mL). The solution was then cooled to 0° C. This was followed by the addition of PPh₃ (1.65 g, 6.29 mmol, 4.0 eq.). The reaction was then allowed to stir at 0° C. for 20 minutes. Compound 11 (951 mg, 1.57 mmol, 1.0 eq.) was then added and the reaction was stirred at 0° C. for 3 hrs. EtOAc and brine were then added, and the layers were separated. The aqueous layer was extracted three times with EtOAc. The combined organic layers were dried over MgSO₄, filtered, and concentrated. Silica gel column chromatography (1:1 Hex/EtOAc) afforded 13 as a white solid (249 mg, 21%).

¹H NMR (500 MHz, Chloroform-d) δ 6.38 (t, J=7.4 Hz, 1H), 5.87 (d, J=8.3 Hz, 1H), 5.79 (d, J=5.4 Hz, 1H), 5.69 (d, J=5.4 Hz, 1H), 4.37-4.33 (m, 1H), 3.76-3.72 (m, 1H), 3.11-3.05 (m, 1H), 2.59-2.50 (m, 2H), 2.31-2.07 (m, 6H), 1.97 (s, 3H), 1.89-1.80 (m, 2H), 1.77-1.69 (m, 2H), 1.62-1.54 (m, 4H), 1.52-1.47 (m, 1H), 1.37 (s, 3H), 1.30 (d, J=14.3 Hz, 1H), 1.21 (s, 9H), 1.15-1.10 (m, 1H), 0.97 (s, 3H), 0.92-0.89 (m, 6H). (50 non-exchangeable protons).

¹³C NMR (125 MHz, Chloroform-d) δ 177.21, 170.33, 167.65, 153.38, 137.27, 127.71, 90.04, 80.09, 74.43, 71.54, 68.27, 49.39, 49.05, 44.78, 39.65, 39.10, 38.95, 37.17, 36.46, 36.21, 36.00, 33.11, 32.43, 30.37, 30.11, 27.08(3C), 26.85, 24.26, 23.05, 20.97, 20.95, 18.15, 16.12.

HRMS(ESI): m/z calc. for C₃₅H₅₂O₈NaBr₂ [M+Na]+: 781.1927. found: 781.1938.

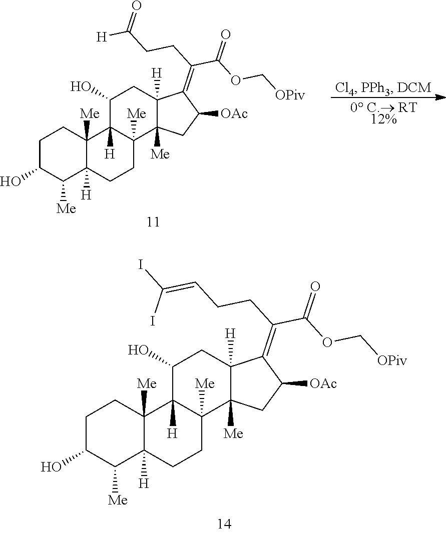

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-diiodohex-5-enoate (14). In a 10 mL round bottom flask, Cl₄ (1.26 g, 2.42 mmol, 2.0 eq.) was dissolved in DCM (22.0 mL). This was followed by the addition of PPh₃ (1.27 g, 4.84 mmol, 4.0 eq.). The reaction was then stirred at room temperature for 30 minutes. Compound 11 (732 mg, 1.21 mmol, 1.0 eq.) was then added and the reaction was stirred at room temperature for 2 hrs. EtOAc and brine were then added, and the layers were separated. The aqueous layer was extracted three times with EtOAc. The combined organic layers were dried over MgSO₄, filtered, and concentrated. Silica gel column chromatography (1:1 Hex/EtOAc) afforded 14 as a white solid (127 mg, 12%).

¹H NMR (500 MHz, Chloroform-d) δ 6.92 (t, J=7.2 Hz, 1H), 5.86 (d, J=8.3 Hz, 1H), 5.78 (d, J=5.4 Hz, 1H), 5.68 (d, J=5.4 Hz, 1H), 4.36-4.32 (m, 1H), 3.73-3.68 (m, 1H), 3.10-3.04 (m, 1H), 2.58-2.46 (m, 2H), 2.29-2.23 (m, 1H), 2.21-2.03 (m, 4H), 1.99-1.97 (m, 1H), 1.96 (s, 3H), 1.86-1.78 (m, 2H), 1.73-1.67 (m, 2H), 1.62-1.51 (m, 3H), 1.50-1.45 (m, 1H), 1.35 (s, 3H), 1.28 (d, J=14.3 Hz, 1H), 1.19 (s, 9H), 1.13-1.06 (m, 2H), 0.95 (s, 3H), 0.90-0.87 (m, 6H). (50 non-exchangeable protons).

¹³C NMR (125 MHz, Chloroform-d) δ 177.16, 170.30, 167.57, 153.49, 151.60, 127.44, 80.08, 74.39, 71.52, 68.13, 49.42, 48.97, 44.79, 39.60, 39.58, 39.01, 38.90, 36.93, 36.65, 36.10, 35.82, 31.98, 30.15, 30.00, 27.08(3C), 26.34, 23.92, 23.36, 21.04, 20.94, 18.03, 16.14, 13.10.

HRMS(ESI): m/z calc. for C₃₅H₅₂O₈NaI₂ [M+Na]+: 877.1649. found: 877.1650.

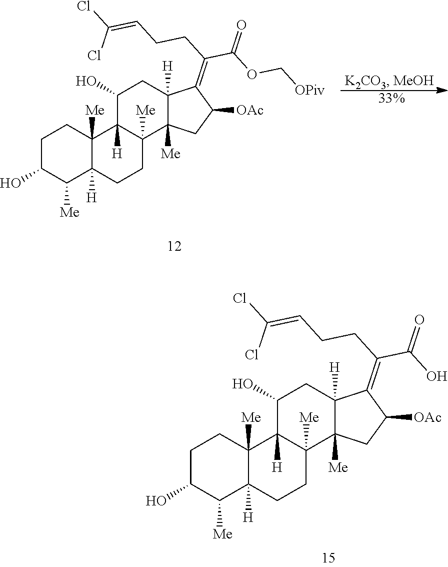

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-dichlorohex-5-enoic acid (15). In a 5 mL vial, 12 (82.4 mg, 0.12 mmol, 1.0 eq.) was dissolved in MeOH (1.39 mL). K₂CO₃ (33.9 mg, 0.24 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 15 (22.7 mg, 33%) as a white solid.

¹H NMR (500 MHz, Methanol-d₄) δ 6.03 (t, J=7.4 Hz, 1H), 5.79 (d, J=8.3 Hz, 1H), 4.32-4.27 (m, 1H), 3.67-3.64 (m, 1H), 3.06-2.99 (m, 1H), 2.66-2.59 (m, 1H), 2.47-2.40 (m, 1H), 2.36-2.23 (m, 4H), 2.20-2.08 (m, 2H), 2.00 (s, 3H), 1.87-1.64 (m, 5H), 1.61 (s, 1H), 1.57-1.44 (m, 2H), 1.39 (s, 3H), 1.20 (d, J=14.0 Hz, 1H), 1.16-1.10 (m, 2H), 0.99 (s, 3H), 0.95 (s, 3H), 0.89 (d, J=6.8 Hz, 3H). (39 non-exchangeable protons).

¹³C NMR (125 MHz, Methanol-d₄) δ 178.94, 173.42, 139.76, 138.09, 131.33, 120.68, 75.94, 72.60, 68.86, 50.78, 50.15, 43.87, 40.77, 40.27, 38.32, 37.89, 37.57, 36.91, 33.00, 31.11, 31.03, 30.68, 29.04, 23.88, 23.87, 22.53, 21.17, 17.96, 16.59.

HRMS(ESI): m/z calc. for C₂₉H₄₂O₆NaCl₂ [M+Na]+: 579.2256. found: 579.2245.

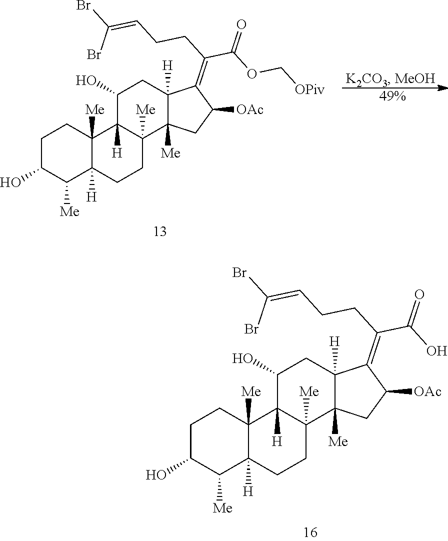

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-dibromohex-5-enoic acid (16). In a 5 mL vial, 13 (48.4 mg, 0.06 mmol, 1.0 eq.) was dissolved in MeOH (0.90 mL). K₂CO₃ (17.5 mg, 0.12 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 16 (20.1 mg, 49%) as a white solid.

¹H NMR (500 MHz, Methanol-d₄) δ 6.55 (t, J=7.4 Hz, 1H), 5.78 (d, J=8.3 Hz, 1H), 4.32-4.28 (m, 1H), 3.67-3.63 (m, 1H), 3.05-2.99 (m, 1H), 2.66-2.58 (m, 1H), 2.49-2.41 (m, 1H), 2.32-2.22 (m, 4H), 2.20-2.08 (m, 2H), 2.00 (s, 3H), 1.85-1.63 (m, 5H), 1.61 (s, 1H), 1.56-1.45 (m, 2H), 1.39 (s, 3H), 1.20 (d, J=14.0 Hz, 1H), 1.17-1.09 (m, 2H), 1.00 (s, 3H), 0.95 (s, 3H), 0.89 (d, J=6.7 Hz, 3H). (39 non-exchangeable protons).

¹³C NMR (125 MHz, Methanol-d₄) δ 178.96, 173.42, 140.05, 139.60, 138.12, 89.28, 75.99, 72.62, 68.89, 50.80, 50.15, 43.84, 40.78, 40.29, 38.32, 37.90, 37.63, 36.93, 34.06, 33.01, 31.12, 31.04, 28.74, 23.87(2C), 22.53, 21.17, 17.98, 16.58.

HRMS(ESI): m/z calc. for C₂₉H₄₂O₆NaBr₂ [M+Na]+: 667.1246. found: 667.1255.

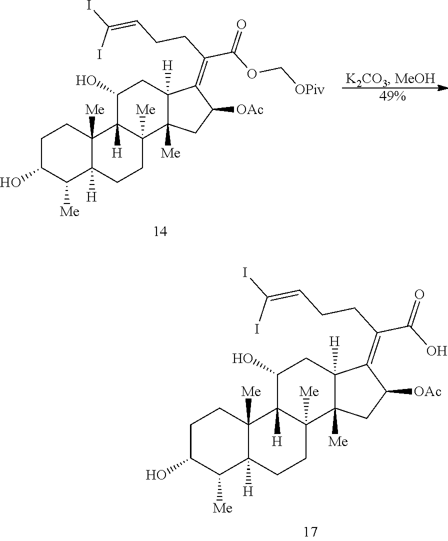

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-diiodohex-5-enoic acid (17). In a 5 mL vial, 14 (127 mg, 0.15 mmol, 1.0 eq.) was dissolved in MeOH (1.70 mL). K₂CO₃ (41.3 mg, 0.30 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 17 (54.0 mg, 49%) as a white solid.

¹H NMR (500 MHz, Methanol-d₄) δ 7.05 (t, J=7.2 Hz, 1H), 5.79 (d, J=8.3 Hz, 1H), 4.34-4.29 (m, 1H), 3.68-3.64 (m, 1H), 3.05-3.00 (m, 1H), 2.64-2.57 (m, 1H), 2.48-2.41 (m, 1H), 2.34-2.23 (m, 2H), 2.19-2.07 (m, 4H), 2.00 (s, 3H), 1.88-1.80 (m, 2H), 1.79-1.63 (m, 3H), 1.61 (s, 1H), 1.55-1.47 (m, 2H), 1.39 (s, 3H), 1.19 (d, J=14.0 Hz, 1H), 1.16-1.10 (m, 2H), 1.00 (s, 3H), 0.96 (s, 3H), 0.89 (d, J=6.8 Hz, 3H). (39 non-exchangeable protons).

¹³C NMR (125 MHz, Methanol-d₄) δ 178.91, 173.42, 154.06, 139.66, 138.02, 75.90, 72.57, 68.85, 50.76, 50.16, 43.86, 40.74, 40.59, 40.26, 38.30, 37.88, 37.76, 36.90, 33.00, 31.11, 31.01, 28.36, 23.91, 23.86, 22.52, 21.18, 18.02, 16.59, 12.96.

HRMS(ESI): m/z calc. for C₂₉H₄₂O₆NaI₂ [M+Na]+: 763.0969. found: 763.0955.

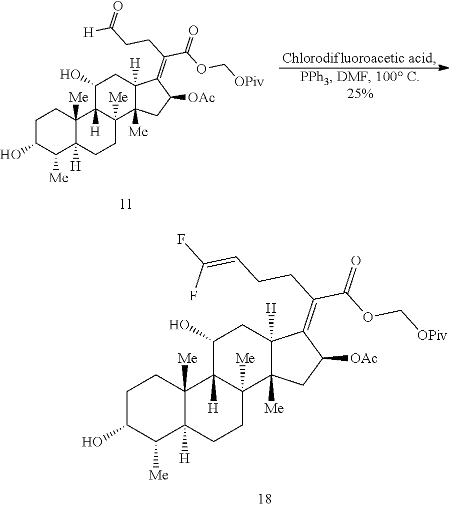

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-difluorohex-5-enoate (18). In a 5 mL vial, 11 (104 mg, 0.17 mmol, 2.0 eq.) was dissolved in DMF (1.00 mL). This was followed by the addition of PPh₃ (90.5 mg, 0.34 mmol, 2.0 eq.) and chlorodifluoroacetic acid (52.6 mg, 0.34 mmol, 2.0 eq.). The reaction was then heated to 100° C. for 4 hrs. EtOAc and brine were then added, and the layers were separated. The aqueous layer was extracted three times with EtOAc. The combined organic layers were dried over MgSO₄, filtered, and concentrated. Silica gel column chromatography (1:1 Hex/EtOAc) afforded 18 as a white solid (27.8 mg, 25%).

¹H NMR (500 MHz, MeOD) δ 5.85 (d, J=8.3 Hz, 1H), 5.77 (d, J=5.7 Hz, 1H), 5.72 (d, J=5.7 Hz, 1H), 4.33-4.31 (m, 1H), 4.31-4.23 (m, 1H), 3.68-3.65 (m, 1H), 3.14-3.09 (m, 1H), 2.63-2.56 (m, 1H), 2.52-2.45 (m, 1H), 2.31-2.24 (m, 2H), 2.21-2.12 (m, 3H), 2.10-2.02 (m, 1H), 1.96 (s, 3H), 1.91-1.80 (m, 2H), 1.77-1.62 (m, 3H), 1.61-1.58 (m, 1H), 1.56-1.51 (m, 1H), 1.50-1.46 (m, 1H), 1.40 (s, 3H), 1.27 (d, J=14.3 Hz, 1H), 1.21 (s, 9H), 1.18-1.10 (m, 2H), 1.00 (s, 3H), 0.94 (s, 3H), 0.90 (d, J=6.8 Hz, 3H). (50 non-exchangeable protons).

¹³C NMR (125 MHz, MeOD) δ 178.39, 172.13, 169.30, 157.96 (dd, J=284.8, 282.7 Hz, 1C), 154.32, 129.41, 81.15, 78.18 (dd, J=22.5, 20.5, Hz 1C), 75.71, 72.50, 68.54, 50.68, 50.14, 45.85, 40.79, 40.07, 39.86, 38.26, 37.90, 37.35, 36.89, 32.95, 31.10, 31.04, 29.24, 27.33(3C), 24.00, 23.87, 23.65 (d, J=4.3 Hz, 1C), 22.41, 20.95, 18.25, 16.57.

HRMS(ESI): m/z calc. for C₃₅H₅₂O₈NaF₂ [M+Na]+: 661.3528. found: 661.3506.

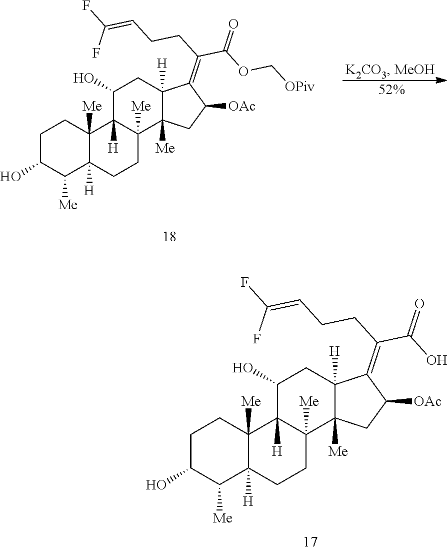

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6,6-difluorohex-5-enoic acid (19). In a 5 mL vial, 18 (89.6 mg, 0.14 mmol, 1.0 eq.) was dissolved in MeOH (1.60 mL). K₂CO₃ (38.7 mg, 0.28 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 19 (38.0 mg, 52%) as a white solid.

¹H NMR (500 MHz, Methanol-d₄) δ 5.78 (d, J=8.3 Hz, 1H), 4.38-4.31 (m, 1H), 4.30-4.28 (m, 1H), 3.67-3.63 (m, 1H), 3.04-2.99 (m, 1H), 2.64-2.57 (m, 1H), 2.44-2.36 (m, 1H), 2.30-2.23 (m, 2H), 2.20-2.08 (m, 4H), 2.00 (s, 3H), 1.88-1.79 (m, 2H), 1.79-1.62 (m, 3H), 1.62-1.60 (m, 1H), 1.55-1.50 (m, 1H), 1.50-1.45 (m, 1H), 1.38 (s, 3H), 1.19 (d, J=14.0 Hz, 1H), 1.16-1.08 (m, 2H), 0.99 (s, 3H), 0.94 (s, 3H), 0.89 (d, J=6.8 Hz, 3H). (39 non-exchangeable protons).

¹³C NMR (125 MHz, Methanol-d₄) δ 179.22, 173.44, 159.67 (dd, J=283.6, 281.7 Hz, 1C), 139.34, 138.54, 79.11 (dd, J=21.6, 20.7 Hz, 1C), 75.97, 72.62, 68.89, 50.80, 50.12, 43.80, 40.77, 40.29, 38.33, 37.89, 37.48, 36.91, 32.99, 31.11, 31.03, 30.52, 23.89, 23.86, 23.25 (d, J=4.1 Hz 1C), 22.54, 21.18, 17.94, 16.58.

HRMS(ESI): m/z calc. for C₂₉H₄₂O₆NaF₂[M]−: 523.2871. found: 523.2872.

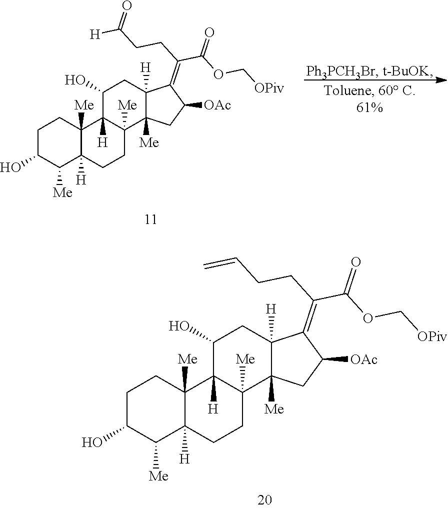

Experimental procedure and spectra for compound 20 have been previously reported (CN105924488).

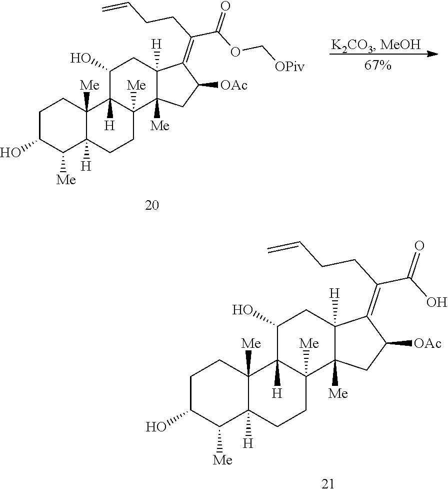

Experimental procedure and spectra for compound 21 have been previously reported (CN105924488).

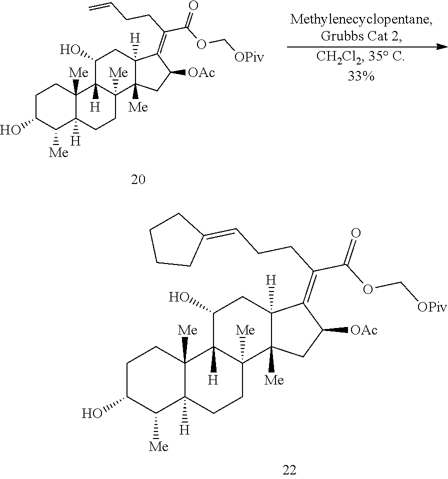

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-cyclopentylidenepentanoate (22). In a 5 mL vial, 20 (158 mg, 0.26 mmol, 1.0 eq.) and Grubbs Cat 2 (13.8 mg, 0.015 mmol, 0.06 eq.) were added. The vial was then degassed and backfilled with nitrogen three times. DCM (1.60 mL) and methylenecyclopentane (0.11 mL, 1.04 mmol, 4.0 eq.) were then added, respectively. The reaction was then heated to 35° C. for 24 hours. EtOAc and brine were then added, and the layers were separated. The aqueous layer was extracted three times with EtOAc. The combined organic layers were dried over MgSO₄, filtered, and concentrated. Silica gel column chromatography (3:1 Hex/EtOAc to 1:1 Hex/EtOAc) afforded 22 as a white solid (57.5 mg, 33%).

¹H NMR (500 MHz, Chloroform-d) 5.83 (d, J=8.3 Hz, 1H), 5.77 (d, J=5.4, 1.3 Hz, 1H), 5.69 (d, J=5.4, 1.3 Hz, 1H), 5.21-5.14 (m, 1H), 4.34-4.29 (m, 1H), 3.73-3.70 (m, 1H), 3.06-3.00 (m, 1H), 2.49-2.40 (m, 2H), 2.33-2.27 (m, 1H), 2.19-2.09 (m, 7H), 2.03-1.97 (m, 1H), 1.96 (s, 3H), 1.84-1.72 (m, 5H), 1.64-1.52 (m, 7H), 1.51-1.46 (m, 1H), 1.35 (s, 3H), 1.28 (d, J=14.3 Hz, 1H), 1.19 (s, 9H), 1.14-1.05 (m, 2H), 0.96 (s, 3H), 0.91-0.88 (m, 6H). (58 non-exchangeable protons).

¹³C NMR (125 MHz, Chloroform-d) δ 177.17, 170.40, 168.30, 151.02, 144.58, 129.43, 118.50, 79.92, 74.45, 71.52, 68.34, 49.42, 48.89, 44.46, 39.61, 39.12, 38.89, 37.08, 36.52, 36.09, 35.73, 33.73, 32.29, 30.30, 30.07, 29.92, 28.83, 28.72, 27.03(3C), 26.50(2C), 24.10, 23.12, 20.99, 20.95, 18.00, 16.10.

HRMS(ESI): m/z calc. for C₃₉H₆₀O₈Na [M+Na]+: 679.4186. found: 679.4182.

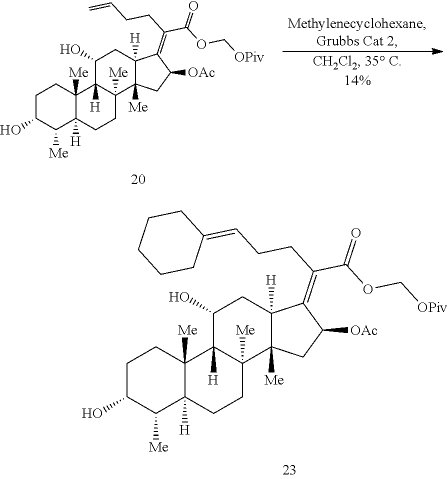

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-cyclohexylidenepentanoate (23). In a 5 mL vial, 20 (140 mg, 0.23 mmol, 1.0 eq.) and Grubbs Cat 2 (11.9 mg, 0.013 mmol, 0.06 eq) were added. The vial was then degassed and backfilled with nitrogen three times. DCM (1.42 mL) and methylenecyclohexane (0.11 mL, 0.93 mmol, 4.0 eq.) were then added, respectively. The reaction was then heated to 35° C. for 24 hours. EtOAc and brine were then added, and the layers were separated. The aqueous layer was extracted three times with EtOAc. The combined organic layers were dried over MgSO₄, filtered, and concentrated. Silica gel column chromatography (3:1 Hex/EtOAc to 1:1 Hex/EtOAc) afforded 23 as a white solid (22.0 mg, 14%).

¹H NMR (500 MHz, Chloroform-d) δ 5.85 (d, J=8.3 Hz, 1H), 5.79 (d, J=5.4, 1.3 Hz, 1H), 5.69 (d, J=5.4, 1.3 Hz, 1H), 5.06-5.00 (m, 1H), 4.36-4.31 (m, 1H), 3.76-3.71 (m, 1H), 3.07-3.00 (m, 1H), 2.50-2.38 (m, 2H), 2.35-2.29 (m, 1H), 2.20-2.02 (m, 8H), 1.97 (s, 3H), 1.89-1.79 (m, 2H), 1.78-1.70 (m, 2H), 1.60-1.47 (m, 11H), 1.36 (s, 3H), 1.29 (d, J=14.3 Hz, 1H), 1.20 (s, 9H), 1.16-1.04 (m, 2H), 0.97 (s, 3H), 0.93-0.88 (m, 6H). (60 non-exchangeable protons).

¹³C NMR (125 MHz, Chloroform-d) δ 177.19, 170.42, 168.32, 151.05, 140.95, 129.45, 119.68, 79.93, 74.48, 71.53, 68.45, 49.38, 48.94, 44.49, 39.63, 39.16, 38.93, 37.33, 37.22, 36.39, 36.33, 35.79, 32.57, 30.43, 30.14, 29.23, 28.91, 28.71, 28.03, 27.47, 27.06(4C), 24.32, 22.93, 20.99, 20.93, 18.11, 16.11.

HRMS(ESI): m/z calc. for C₄₀H₆₂O₈Na [M+Na]+: 693.4342. found: 693.4344.

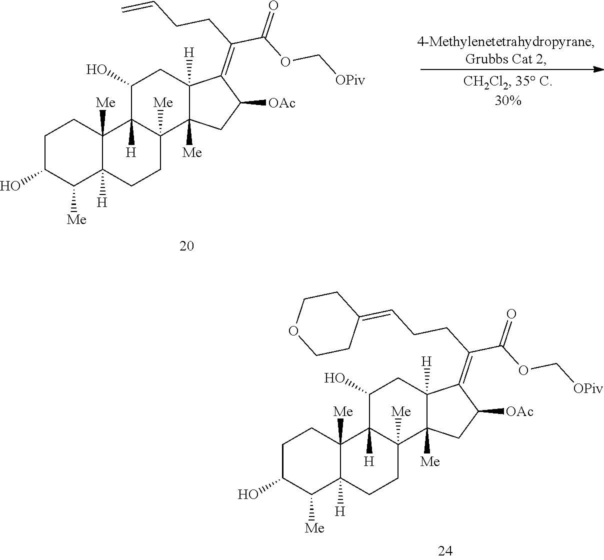

Procedure for (pivaloyloxy)methyl (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-(tetrahydro-4H-pyran-4-ylidene)pentanoate (24). In a 5 mL vial, 20 (220 mg, 0.36 mmol, 1.0 eq.) and Grubbs Cat 2 (18.5 mg, 0.021 mmol, 0.06 eq) were added. The vial was then degassed and backfilled with nitrogen three times. DCM (2.20 mL) and 4-methylenetetrahydropyrane (0.16 mL, 1.45 mmol, 4.0 eq.) were then added, respectively. The reaction was then heated to 35° C. for 24 hours. EtOAc and brine were then added, and the layers were separated. The aqueous layer was extracted three times with EtOAc. The combined organic layers were dried over MgSO₄, filtered, and concentrated. Silica gel column chromatography (1:1 Hex/EtOAc to 100% EtOAc) afforded 24 as a white solid (73.7 mg, 30%).

¹H NMR (500 MHz, Chloroform-d) δ 5.83 (d, J=8.3 Hz, 1H), 5.76 (d, J=5.4 Hz, 1H), 5.67 (d, J=5.4 Hz, 1H), 5.17-5.11 (m, 1H), 4.33-4.29 (m, 1H), 3.73-3.70 (m, 1H), 3.67-3.55 (m, 4H), 3.05-3.00 (m, 1H), 2.48-2.41 (m, 2H), 2.33-2.26 (m, 1H), 2.22-2.08 (m, 8H), 1.95 (s, 3H), 1.86-1.70 (m, 5H), 1.60-1.52 (m, 3H), 1.50-1.45 (m, 1H), 1.34 (s, 3H), 1.27 (d, J=14.3 Hz, 1H), 1.18 (s, 9H), 1.14-1.06 (m, 2H), 0.95 (s, 3H), 0.90-0.87 (m, 6H). (58 non-exchangeable protons).

¹³C NMR (125 MHz, Chloroform-d) δ 177.16, 170.37, 168.23, 151.45, 135.64, 129.12, 121.66, 79.92, 74.42, 71.48, 69.69, 68.97, 68.38, 49.39, 48.94, 44.56, 39.62, 39.13, 38.91, 37.20, 37.06, 36.36, 36.27, 35.67, 32.53, 30.44, 30.10, 29.89, 29.00, 27.23, 27.05(3C), 24.33, 22.95, 20.97, 20.90, 18.11, 16.11.

HRMS(ESI): m/z calc. for C₃₉H₆₀O₉Na [M+Na]+: 695.4135. found: 695.4134.

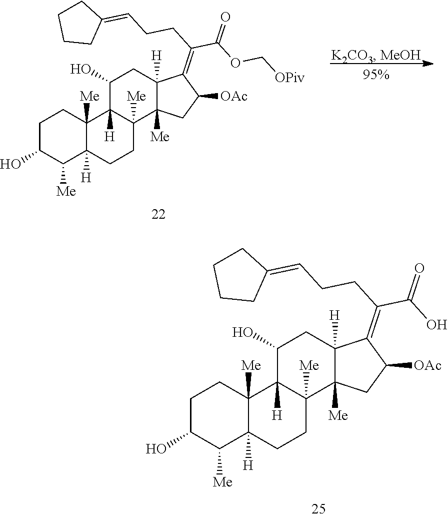

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-cyclopentylidenepentanoic acid (25). In a 5 mL vial, 22 (139 mg, 0.21 mmol, 1.0 eq.) was dissolved in MeOH (2.42 mL). K₂CO₃ (58.7 mg, 0.42 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 25 (110 mg, 95%) as a white solid.

¹H NMR (500 MHz, Methanol-d₄) δ 5.77 (d, J=8.3 Hz, 1H), 5.30-5.23 (m, 1H), 4.31-4.28 (m, 1H), 3.67-3.63 (m, 1H), 3.03-2.97 (m, 1H), 2.59-2.52 (m, 1H), 2.37-2.25 (m, 3H), 2.24-2.17 (m, 4H), 2.17-2.07 (m, 4H), 2.00 (s, 3H), 1.87-1.69 (m, 4H), 1.68-1.56 (m, 6H), 1.56-1.46 (m, 2H), 1.38 (s, 3H), 1.19 (d, J=14.3 Hz, 1H), 1.16-1.09 (m, 2H), 0.99 (s, 3H), 0.95 (s, 3H), 0.89 (d, J=6.8 Hz, 3H). (47 non-exchangeable protons).

¹³C NMR (125 MHz, Methanol-d₄) δ 179.70, 173.54, 144.12, 139.81, 137.90, 121.06, 76.08, 72.62, 68.96, 50.83, 50.06, 43.75, 40.77, 40.36, 38.33, 37.89, 37.51, 36.92, 34.63, 33.01, 31.11, 31.04, 30.90, 30.71, 29.58, 27.53, 27.49, 23.89, 23.86, 22.55, 21.23, 17.96, 16.58.

HRMS(ESI): m/z calc. for C₃₃H₅₀O₆Na [M+Na]+: 565.3505. found: 565.3503.

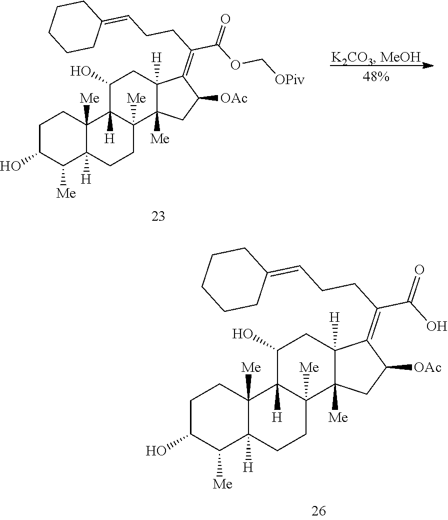

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-cyclohexylidenepentanoic acid (26). In a 5 mL vial, 23 (75.5 mg, 0.11 mmol, 1.0 eq.) was dissolved in MeOH (1.28 mL). K₂CO₃ (31.1 mg, 0.22 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 26 (29.9 mg, 48%) as a white solid.

¹H NMR (500 MHz, Methanol-d₄) δ 5.76 (d, J=8.3 Hz, 1H), 5.13-5.08 (m, 1H), 4.31-4.28 (m, 1H), 3.66-3.64 (m, 1H), 3.03-2.97 (m, 1H), 2.54-2.48 (m, 1H), 2.33-2.22 (m, 3H), 2.19-2.09 (m, 6H), 2.08-2.04 (m, 2H), 2.00 (s, 3H), 1.85-1.67 (m, 4H), 1.65-1.59 (m, 2H), 1.57-1.47 (m, 8H), 1.38 (s, 3H), 1.19 (d, J=14.0 Hz, 1H), 1.16-1.09 (m, 2H), 0.99 (s, 3H), 0.95 (s, 3H), 0.89 (d, J=6.8 Hz, 3H). (49 non-exchangeable protons).

¹³C NMR (125 MHz, Methanol-d₄) δ 179.67, 173.50, 140.74, 139.47, 138.11, 122.24, 76.06, 72.63, 68.96, 50.80, 50.05, 43.73, 40.75, 40.32, 38.34, 38.32, 37.86, 37.45, 36.86, 32.98, 31.55, 31.09, 31.01, 29.94, 29.83, 29.13, 28.38, 28.19, 23.92, 23.86, 22.56, 21.21, 17.93, 16.61.

HRMS(ESI): m/z calc. for C₃₄H₅₂O₆Na [M+Na]+: 579.3662. found: 579.3660.

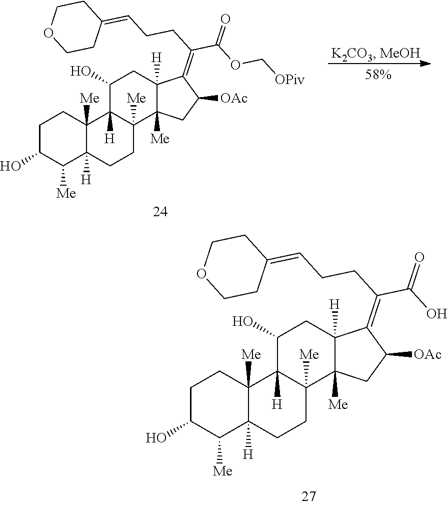

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-5-(tetrahydro-4H-pyran-4-ylidene)pentanoic acid (27). In a 5 mL vial, 24 (75.1 mg, 0.11 mmol, 1.0 eq.) was dissolved in MeOH (1.27 mL). K₂CO₃ (30.8 mg, 0.22 mmol, 2.0 eq.) was added to the reaction. The reaction was then stirred at room temperature for 1 hr. The reaction was then filtered and concentrated. The resulting crude product was then purified by preparatory HPLC (C18 20×250 mm, 10-100% ACN in H₂O) to afford 27 (36.0 mg, 58%) as a white solid.

¹H NMR (500 MHz, Methanol-d₄) δ 5.78 (d, J=8.3 Hz, 1H), 5.30-5.24 (m, 1H), 4.30-4.28 (m, 1H), 3.67-3.61 (m, 5H), 3.03-2.97 (m, 1H), 2.58-2.50 (m, 1H), 2.36-2.26 (m, 5H), 2.20-2.07 (m, 6H), 2.00 (s, 3H), 1.87-1.65 (m, 4H), 1.65-1.60 (m, 2H), 1.55-1.51 (m, 1H), 1.50-1.46 (m, 1H), 1.38 (s, 3H), 1.19 (d, J=14.3 Hz, 1H), 1.16-1.10 (m, 2H), 0.99 (s, 3H), 0.95 (s, 3H), 0.89 (d, J=6.8 Hz, 3H). (47 non-exchangeable protons).

¹³C NMR (125 MHz, Methanol-d₄) δ 179.54, 173.51, 139.42, 138.28, 135.42, 124.42, 76.05, 72.60, 70.84, 70.10, 68.94, 50.83, 50.09, 43.79, 40.78, 40.33, 38.31, 38.03, 37.89, 37.51, 36.92, 33.01, 31.27, 31.11, 31.05, 30.82, 28.09, 23.88(2C), 22.53, 21.22, 17.97, 16.59.

HRMS(ESI): m/z calc. for C₃₃H₅₀O₇Na [M+Na]+: 581.3454. found: 581.3452.

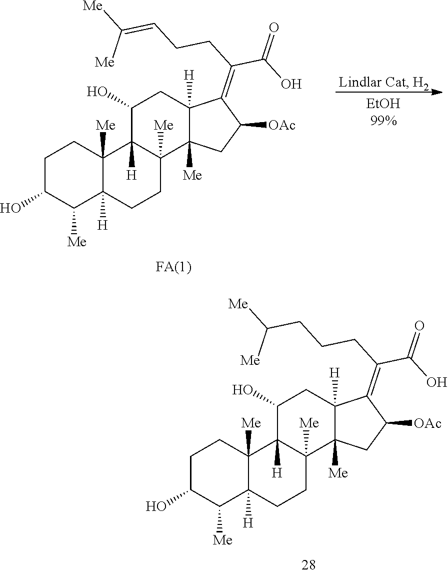

Procedure for (Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6-methylheptanoic acid (28). In a 25 mL RB Flask, FA (200 mg, 0.387 mmol, 1.0 eq.) was dissolved in EtOH (10.0 mL). Then the solution was degassed with nitrogen. Next, 5% Pd with CaCO₃ (179 mg, 0.29 mmol, 0.09 eq.) was added, and the reaction was degassed again with nitrogen. The reaction was stirred under a hydrogen atmosphere for 2 hours. The reaction was then filtered. The resulting crude product was then purified by preparatory HPLC (C18 20×250 mm, 10-100% ACN in H₂O) to afford 28 (198 mg, 99%) as a white solid.

¹H NMR (500 MHz, Chloroform-d) δ 5.89 (d, J=8.3 Hz, 1H), 4.38-4.32 (m, 1H), 3.79-3.71 (m, 1H), 3.07-3.01 (m, 1H), 2.46-2.33 (m, 2H), 2.30-2.25 (m, 1H), 2.20-2.08 (m, 3H), 1.97 (s, 3H), 1.90-1.81 (m, 2H), 1.78-1.71 (m, 2H), 1.62-1.55 (m, 3H), 1.53-1.43 (m, 3H), 1.37 (s, 3H), 1.35-1.31 (m, 1H), 1.29 (d, J=14.3 Hz, 1H), 1.20-1.05 (m, 4H), 0.97 (s, 3H), 0.93-0.90 (m, 6H), 0.86 (d, J=6.6 Hz, 6H). (47 non-exchangeable protons).

¹³C NMR (125 MHz, Chloroform-d) δ 174.56, 171.03, 149.45, 130.79, 74.52, 71.64, 68.50, 49.43, 48.91, 44.23, 39.64, 39.16, 38.94, 37.22, 36.43, 36.33, 35.64, 32.52, 30.43, 30.07, 29.08, 28.01, 27.90, 24.29, 23.00, 22.80, 22.76, 20.98, 20.84, 18.04, 16.10.

HRMS(ESI): m/z calc. for C₃₁H₅₀O₆[M+Na]+: 541.3505. found: 541.3514.

### Example 4. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A compound of Formula I: 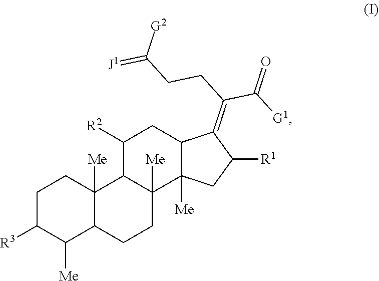
or a salt thereof;
wherein
G¹ is OR^{x}, H, NHOH, NH₂, imidazole, triazole, tetrazole, wherein R^{x} is H or a protecting group;
G² is H, halo, OH, —(C₁-C₆)alkyl, —O(C₁-C₆)alkyl, or NR^{a}R^{b} wherein R^{a} and R^{b} are each independently H, —(C₁-C₆)alkyl, —(C₃-C₆)cycloalkyl, or aryl;
J¹ is CR^{c}R^{d}, O, or absent, wherein G₂ is NR^{a}R^{b} when J¹ is absent, or G₂ is OH or —O(C₁-C₆)alkyl when J₁ is O;
R^{c} and R^{d} taken together form a cycloalkyl or heterocycloalkyl, wherein a ring bond of cycloalkyl or heterocycloalkyl is optionally an endocyclic double bond; or
R^{c} and R^{d} are each independently H, halo, aryl, —(C₁-C₆)alkyl, —(C₃-C₆)cycloalkyl, or —(C₀-C₅)R^{e} wherein R^{e} is OH, —O(C₁-C₆)alkyl, or —C(═O)O(C₁-C₆)alkyl, wherein R^{c} and R^{d} are not both H or not both methyl;
R¹ is —OC(═O)(C₁-C₆)alkyl. H, methyl, ethyl, hydroxy, methoxy, ethoxy, or amino; and
R², and R³ are each independently hydroxy, H, methyl, ethyl, methoxy, ethoxy, amino, or —OC(═O)(C₁-C₆)alkyl.

2. The compound of claim 1 wherein the compound is: 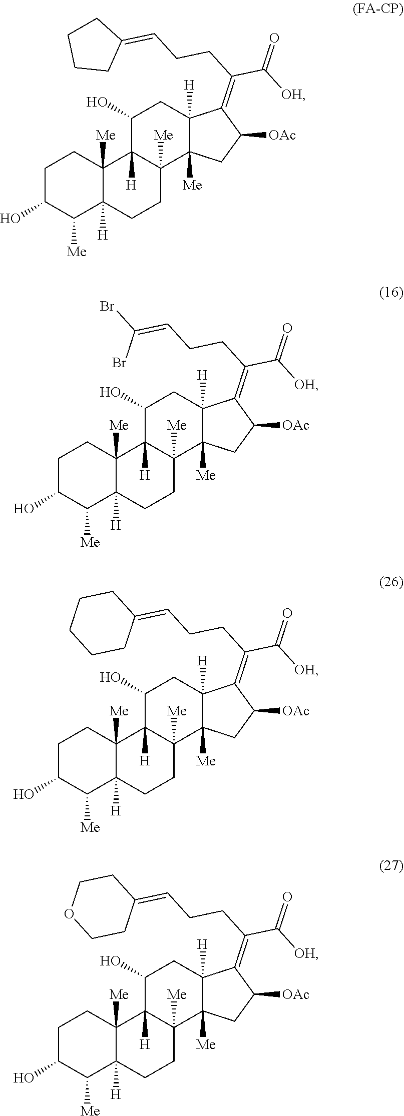
or a salt thereof.

3. The compound of claim 1 wherein G¹ is OR^{x}.

4. The compound of claim 3 wherein OR^{x} is OH.

5. The compound of claim 1 wherein G² is H.

6. The compound of claim 1 wherein J¹ is CR^{c}R^{d}.

7. The compound of claim 6 wherein R^{c} and R^{d} taken together form a cycloalkyl or heterocycloalkyl.

8. The compound of claim 6 wherein J¹ is: 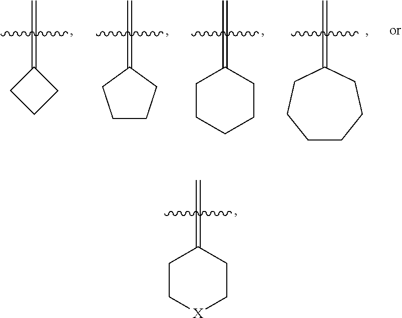
wherein X is O or NR^{f} and R^{f} is H, —(C₁-C₆)alkyl, —(C₃-C₆)cycloalkyl, or aryl.

9. The compound of claim 6 wherein J¹ is:

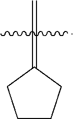 [SMILES: *=C1CCCC1]

10. The compound of claim 6 wherein J₁ is:

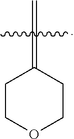 [SMILES: *=C1CCOCC1]

11. The compound of claim 6 wherein R^{c} and R^{d} are halo.

12. The compound of claim 11 wherein halo is bromo.

13. The compound of claim 11 wherein halo is chloro or iodo.

14. The compound of claim 6 wherein R^{c} is —(C₁-C₆)alkyl and R^{d} is —(C₀-C₅)R^{e}.

15. The compound of claim 14 wherein R^{d} is —(C₀-C₅)O(C₁-C₆)alkyl.

16. The compound of claim 1 wherein the compound is:

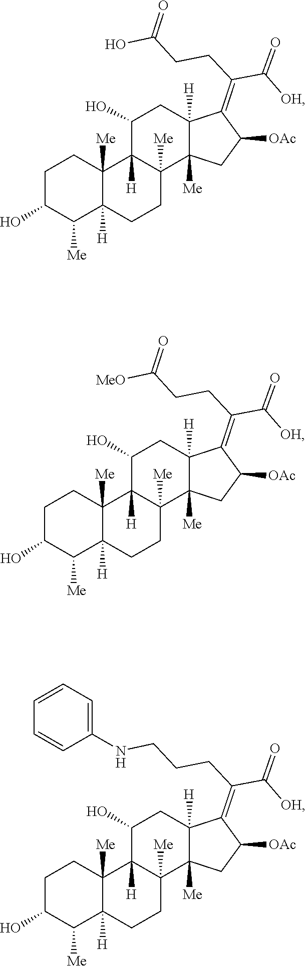

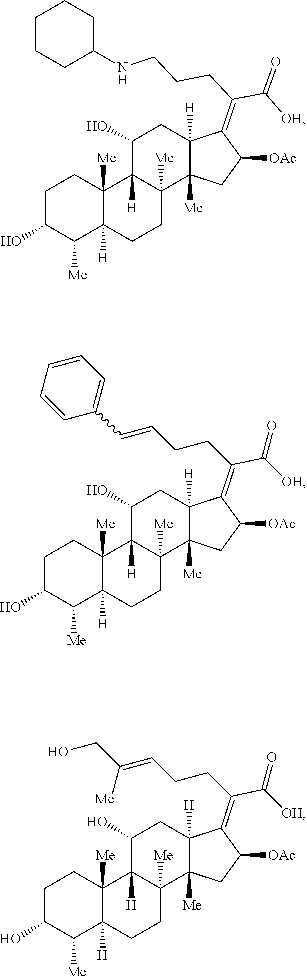

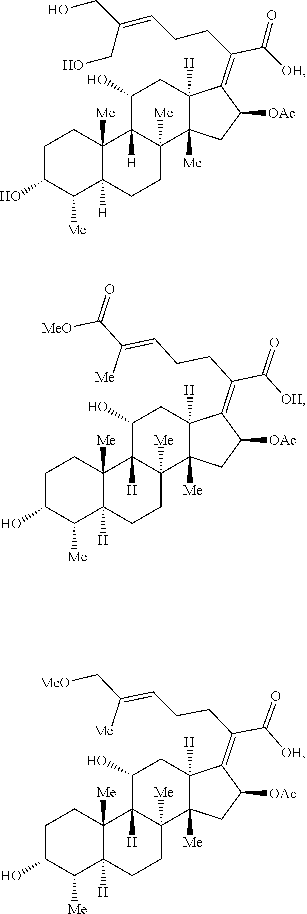

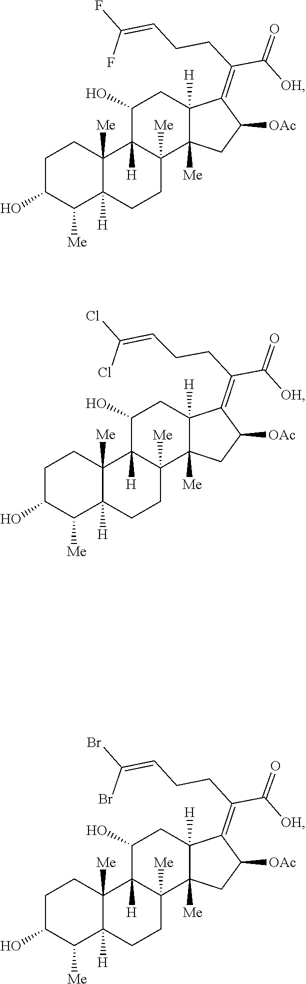

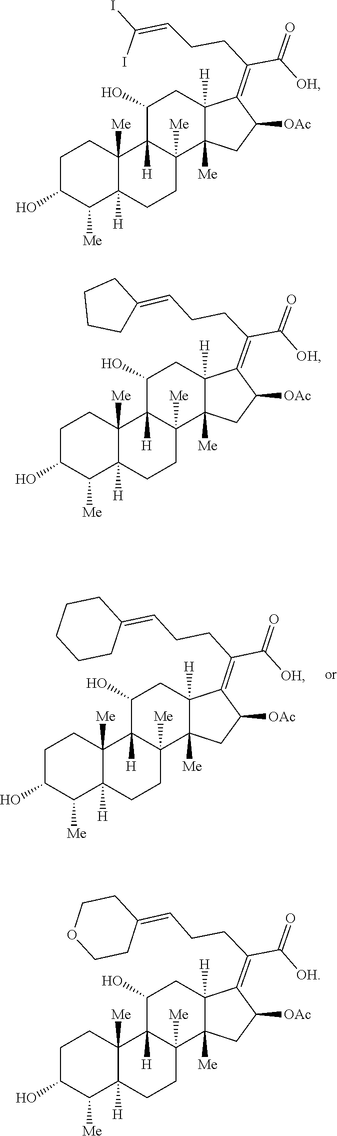

17. The compound of claim 1 wherein the compound is:

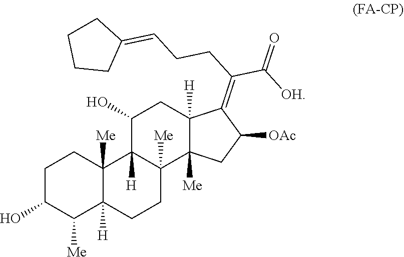

18. A pharmaceutical composition comprising a compound of claim 1 and a pharmaceutically acceptable excipient.

19. A method of antimicrobial treatment comprising administering to a subject in need thereof a therapeutically effective amount of a compound of claim 1 or a pharmaceutically acceptable salt thereof, thereby killing or inhibiting the growth of at least a portion of a plurality of microorganisms in the subject.

20. The method of claim 19 wherein the microorganism is a Gram-negative bacterium.

21. The method of claim 19 wherein the microorganism is Acinetobacter, anthrax-causing bacteria, Bacilli, Bordetella, Borrelia, botulism-causing bacteria, Brucella, Burkholderia, Campylobacter, Chlamydia, cholera-causing bacteria, Clostridium, Conococcus, Corynebacterium, diptheria-causing bacteria, Enterobacter, Enterococcus, Erwinia, Escherichia, Francisella, Haemophilus, Heliobacter, Klebsiella, Legionella, Leptospira, leptospirosis-causing bacteria, Listeria, Lyme's disease-causing bacteria, meningococcus, Mycobacterium, Mycoplasma, Neisseria, Pasteurella, Pelobacter, plague-causing bacteria, Pneumonococcus, Proteus, Pseudomonas, Rickettsia, Salmonella, Serratia, Shigella, Staphylococcus, Streptococcus, tetanus, Treponema, Vibrio, Yersinia, Xanthomonas, or a combination thereof.
